(19) United States
(12) Patent Application Publication
(10) Pub. No.: US 2007/0037779 A1
(43) Pub. Date: Feb. 15, 2007

(54) Prevention of thrombotic disorders with active vitamin D compounds or mimics thereof

(76) Inventors: John G. Curd, Hillsborough, CA (US); William David Henner, Portland, OR (US); Tomasz M. Beer, Portland, OR (US); Bradford S. Goodwin, San Mateo, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/482,111

(22) Filed: Jul. 7, 2006

Related U.S. Application Data
(63) Continuation-in-part of application No. PCT/US06/00181, filed on Jan. 5, 2006.
(60) Provisional application No. 60/641,137, filed on Jan. 5, 2005.
(60) Provisional application No. 60/721,130, filed on Sep. 28, 2005.

(51) Int. Cl. A61K 31/59; A61K 31/355

(52) U.S. Cl. 514/167; 514/458

(57) ABSTRACT

The present invention relates to a method for preventing, treating, or ameliorating thrombotic disorders in an animal comprising administering to the animal an active vitamin D compound or a mimic thereof. According to the invention, the active vitamin D compound or the mimic thereof may be administered by HDPA so that high doses of the active vitamin D compound or the mimic thereof can be administered to an animal without inducing severe symptomatic hypercalcemia. The invention also relates a method for preventing, treating, or ameliorating thrombotic disorders in an animal comprising administering to the animal an active vitamin D compound or a mimic thereof in combination with one or more other therapeutic agents.

### BACKGROUND OF THE INVENTION

1. Field of the Invention

The present invention relates to a method for preventing, treating, or ameliorating thrombotic disorders in an animal by administering to the animal active vitamin D compounds or mimics thereof. The invention further relates to a method for preventing, treating, or ameliorating thrombotic disorders in an animal by administering to the animal active vitamin D compounds or mimics thereof in combination with other therapeutic agents.

2. Related Art

Blood coagulation is a process that changes circulating substances within the blood into an insoluble gel. The gel plugs leaks in blood vessels and stops the loss of blood. The process requires coagulation factors, which are biosynthesized by the liver and numbered in the order of their discovery. There are 13 numerals but only 12 factors. Factor VI was subsequently found to be part of another factor. The following are coagulation factors and their common names:

Factor I—fibrinogen

Factor II—prothrombin

Factor III—tissue thromboplastin (tissue factor)

Factor IV—ionized calcium (Ca⁺⁺)

Factor V—labile factor or proaccelerin

Factor VI—unassigned

Factor VII—stable factor or proconvertin (autoprothrombin I)

Factor VIII—antihemophilic factor

Factor IX—plasma thromboplastin component, Christmas

Factor X—Stuart-Prower factor

Factor XI—plasma thromboplastin antecedent

Factor XII—Hageman factor

Factor XIII—fibrin-stabilizing factor

Normal hemostasis is initiated when blood is exposed to subendothelial connective tissues as a result of disruption of the vascular endothelial lining. Within seconds of activating the hemostatic system, platelets are recruited to the injury site forming a platelet plug, which stops blood loss from capillaries, small arterioles, and venules. The recruited platelets adhere to collagen fibrils in vascular subendothelium via a specific platelet collagen receptor, glycoprotein Ia/IIa, which is a member of integrin family. An adhesive glycoprotein called von Willebrand factor allows platelets to remain attached to the vessel wall despite the high shear forces generated within the vascular lumen. Sixma, F. F., “Role of blood platelets, plasma proteins and the vessel wall in haemostasis,” in Haemostasis and Thrombosis, Bloom, A. L. and D. P. Thomas (eds.) Churchill Livingstone, Edinburgh, UK, 2^{nd} ed., 1987.

Exposure of the blood plasma to protein tissue factor (“TF”) on subendothelial connective tissue cells also initiates a cascade of events that activate coagulation factors, which are protease zymogens. The coagulation cascade starts with the tissue factor activating a few molecules of Factor VII, which activate molecules of Factor X, which activate prothrombin, leading to the formation of thrombin. Thrombin, a serine protease, is a potent physiologic mediator of platelet generation and is generated in a manner independent of the initiating platelet agonist. Further, thrombin generation on platelet surface is catalyzed by enzyme-cofactor complex while its action towards platelet receptor is mediated by enzymatic proteolysis. For each thrombin molecule generated, a large number of platelet receptors are activated making thrombin the principle mediator of the platelet-dependent arterial thrombotic process. Thrombin also performs specific cleavages necessary to activate fibrinogen. Activated fibrinogen assembles and polymerizes into large stringy networks, trapping blood cells and forming the dark red scab that blocks the damage.

Presence of TF in circulating blood may also trigger thrombin activation cascade even without injury to the blood vessel. It has been reported that thrombogenic TF is circulating in the blood. Giesen, P. L. et al., “Blood-borne tissue factor: another view of thrombosis,” Proc. Natl. Acad. Sci. 96:2311-15 (1999). Evidence that indicate presence of TF in blood include thrombi formation on perfused pig media, which displays intense staining for TF, whereas the substrate alone did not. Similarly, thrombi deposited on collagen-coated slides display intense staining for TF whereas the substrate alone did not. Moreover, inhibition of circulating TF activity reduces thrombus formation in both media. In addition, Giesen et al. isolated TF and TF-positive neutrophils from whole blood. Thus, the danger for thrombus formation is always present even without exposing the circulating blood to subendothelial connective tissue cells.

Moreover, material with TF activity may enter the blood causing disseminated intravascular coagulation, which is an acquired coagulation disorder. Clinical circumstances that may give rise to TF activity within the blood include complications of obstetrics where uterine material with TF activity gains access to the maternal circulation (e.g., in abruptio placentae, a saline-induced therapeutic abortion, retained dead fetus syndrome, and the initial phase of amniotic fluid embolism). Infections may also lead to TF activity within the blood where gram-negative endotoxins in the blood may cause generation of TF activity on the plasma membrane of monocytes. Certain malignancies, including mucin-secreting adenocarcinomas of the pancreas and prostate and granulocytic leukemia, are also thought to release material with TF activity.

Conversely, endogenous substances that inhibit blood coagulation may also be present in the blood in the form of antibodies that neutralize a clotting factor activity (e.g., an antibody against factor VIII or factor V). For example, in patients with multiple myeloma or other hematologic malignancies, circulating anticoagulants include glycosaminoglycans with heparin-like anticoagulant activity.

Protein C is a vitamin K dependent serine protease and naturally occurring anticoagulant that plays a role in the regulation of hemostasis by inactivating factors V and VIII in the coagulation cascade. Human protein C circulates as a 2-chain zymogen, but functions at the endothelial and platelet surface following conversion to activated protein C by a thrombin-thrombomodulin complex. Activated protein C functions as an important down-regulator of blood coagulation resulting in protection against thrombosis.

Other causes of acquired coagulation disorders include vitamin K deficiency, liver disease and development of circulating anticoagulants, which are usually antibodies to hemostatic factors.

The most prevalent vascular disease states associated with thrombosis are related to platelet dependent narrowing of the blood supply such as atherosclerosis and arteriosclerosis, acute myocardial infarction, chronic stable angina, unstable angina, transient ischemic attacks and strokes, peripheral vascular disease, venous and arterial thrombosis, preeclampsia, embolism, restenosis following angioplasty, carotid endarterectomy, anastomosis of vascular grafts, etc. These conditions represent a variety of disorders thought to be initiated by platelet activation on vessel walls.

Thus, control of thrombin action is important in promoting hemostasis and in limiting thrombosis. Although direct thrombin inhibitors of various structural classes have been identified recently (Tapparelli, C., et al., “Synthetic low-molecular weight thrombin inhibitors: molecular design and pharmacological profile,” Trends Pharmacol. Sci. 14:366-376 (1993); Claeson, G. “Synthetic peptides and peptidomimetics as substrates and inhibitors of thrombin and other proteases in the blood coagulation system,” Blood Coagul. Fibrinolysis 5:411-436 (1994); Lefkovits, J. and Topol, E. J. “Direct thrombin inhibitors in cardiovascular medicine,” Circulation 90(3):1522-1536 (1994)), to date only three classes of compounds (heparins, low-molecular weight heparins and coumarins, such as warfarin) have been used in anticoagulant therapy. Each class has severe limitations and liabilities (Weitz, J. and Hirsh, J. “New anticoagulant strategies,” J. Lab. Clin. Med. 122:364-373 (1993). All three classes indirectly inhibit thrombin. Heparin and low-molecular weight heparins augment anti-thrombin III and/or heparin cofactor II inhibition of thrombin, whereas coumarins inhibit vitamin K-dependent post-translational modifications. Close monitoring and titration of therapeutic doses is required when employing these agents due to patient variability. Hemorrhagic complications due to bleeding are a side effect. In fact, bleeding remains as the most common side effect of long term oral anticoagulant therapy. Lack of activity in arterial thrombosis in the case of heparin is due to its inability to inhibit clot bound thrombin. Lack of oral activity in the case of heparins and low-molecular weight heparins preclude their use for chronic administration

Heparin is administered parenterally in vascular surgery and in the treatment of postoperative thrombosis and embolism Approximately 1 to 30% (average 5%) of patients receiving heparin have an immunologic reaction resulting in heparin-induced thrombocytopenia (HIT) (Phillips, D. E., et al., “Heparin-induced thrombotic thrombocytopenia,” Ann. Pharmacother., 28: 43-45, (1994). These adverse effects may develop into a syndrome known as heparin induced thrombocytopenia and thrombosis syndrome (HITTS). Patients with HITTS are at substantial risk for a debilitating or life-threatening venous or arterial thrombosis, such as lower limb swelling or ischemia, stroke, or myocardial infarction, with a reported combined mortality and major morbidity of 25% to 37% (Boshkov, L. K., et al., “Heparin-induced thrombocytopenia and thrombosis: clinical and laboratory studies,” Br. J. Haemat., 84:322-328, 1993).

Vitamin D is a fat-soluble vitamin essential as a positive regulator of calcium homeostasis. (See Harrison's Principles of Internal Medicine: Part Thirteen, “Disorders of Bone and Mineral Metabolism,” Chapter 353, pp. 2214-2226, A. S. Fauci et al., (eds.), McGraw-Hill, New York (1998)). The hormonally active form of vitamin D is 1α,25-dihydroxyvitamin D₃, also known as calcitriol. Calcitriol is a steroid hormone synthesized from dietary precursors. Dietary 7-dehydrocholesterol is converted to vitamin D₃ by ultraviolet light absorbed through the skin. Vitamin D₃ is hydroxylated at the 25 position by the liver and at the 1 position by the kidneys, converting it to the biologically active form, calcitriol. 1α-hydroxyvitamin D₃, also known as 1α-calcidol, and 25-hydroxyvitamin D₃, also known as calcifediol, are monohydroxylated vitamin D₃ and may be converted to calcitriol upon hydroxylation by the liver and kidney, respectively.

Specific nuclear receptors for active vitamin D compounds have been discovered in cells from diverse organs not involved in calcium homeostasis. (Koyama, T., et al., “Anticoagulant effects of 1α,25-dihydroxyvitamin D₃ on human myelogenous leukemia cells and monocytes,” Blood, 92:160-167 (1998)). Thus, in addition to influencing calcium homeostasis, active vitamin D compounds have been implicated in variety of biological processes including osteogenesis, modulation of immune response, modulation of the process of insulin secretion by the pancreatic B cell, muscle cell function, and the differentiation and growth of epidermal and hematopoietic tissues.

It has been reported that the hormonally active form of vitamin D, calcitriol, exerts anticoagulant effect in vitro by up-regulating the expression of the anticoagulant thrombomodulin (“TM”), and by down-regulating the expression of TF in cultured monocytic cells, including human peripheral monocytes. Koyama, T., et al., Blood, 92:160-167 (1998); Ohsawa, M., et al., “1α,25-Dihydroxyvitamin D₃ and its potent synthetic analogs downregulate tissue factor and upregulate thrombomodulin expression in monocytic cells, counteracting the effects of tumor necrosis factor and oxidized LDL,” Circulation, 102:2867-72 (2000).

Although the administration of active vitamin D compounds may result in substantial therapeutic benefits, the treatment of thrombotic diseases in vivo with such compounds is expected to be limited by the effects these compounds have on calcium metabolism. At the levels shown in vivo for effective use as antithrombotic agents, active vitamin D compounds can induce markedly elevated and potentially dangerous blood calcium levels by virtue of their inherent calcemic activity. That is, the clinical use of calcitriol and other active vitamin D compounds as antithrombotic agents is severely limited by the risk of hypercalcemia.

In connection with the treatment of hyperproliferative diseases, it has been shown that the problem of systemic hypercalcemia can be overcome by “high dose pulse administration” (HDPA) of a sufficient dose of an active vitamin D compound to give an anti-proliferative effect while avoiding the development of severe symptomatic hypercalcemia. According to U.S. Pat. No. 6,521,608, the active vitamin D compound may be administered no more than every three days, for example, once a week at a dose of at least 0.12 μg/kg per day (8.4 μg in a 70 kg person). Pharmaceutical compositions used in the HDPA regimen of U.S. Pat. No. 6,521,608 comprise 5-100 μg of active vitamin D compound and may be administered in the form for oral, intravenous, intramuscular, topical, transdermal, sublingual, intranasal, intratumoral, or other preparations.

### SUMMARY OF THE INVENTION

One aspect of the present invention is a method for preventing, treating, or ameliorating arterial or venous thrombosis in an animal comprising administering to the animal an active vitamin D compound or a mimic thereof. In another embodiment of the invention, the active vitamin D compound, or a mimic thereof, is administered by HDPA so that high doses of the active vitamin D compound or mimic can be administered to an animal without inducing severe symptomatic hypercalcemia. In one aspect, the active vitamin D compound, or a mimic thereof, is administered at a dose of about 0.5 μg to about 300 μg, preferably about 15 μg to about 260 μg, more preferably about 30 μg to about 240 μg, even more preferably about 45 μg to about 220 μg, most preferably about 45 μg to about 200 μg. In another aspect of the invention, the active vitamin D compound or a mimic thereof is administered at a dose sufficient to obtain a peak plasma concentration of the active vitamin D compound or a mimic thereof of at least 0.5 nM. In yet another aspect of the invention, the active vitamin D compound is administered as a unit dosage form comprising about 10 μg to about 75 μg of calcitriol, about 50% MIGLYOL 812 and about 50% tocopherol PEG-1000 succinate (vitamin E TPGS). More preferably, the active vitamin D compound or the mimic thereof is administered as a unit dosage form comprising about 45 μg. The active vitamin D compound or the mimic thereof may be administered orally, intravenously, parenterally, rectally, topically, nasally, sublingually, intramuscularly or transdermally. It is understood that the terms “about 50% MIGLYOL 812” and “about 50% tocopherol PEG-1000 succinate (vitamin E TPGS)” each encompass amounts less than 50% such that one or more active ingredients or other additives may be present in the composition without the composition components totaling more than 100%.

Another aspect of the present invention is a method for preventing, treating, or ameliorating a thrombotic disorder in an animal comprising administering to the animal an active vitamin D compound, or a mimic thereof, in combination with one or more other therapeutic agents, including agents which are a contributing cause of thrombosis and agents which themselves are anti-thrombotic. In one embodiment, the one or more therapeutic agents administered with the active vitamin D compound or the mimic thereof is a chemotherapeutic agent, an anti-angiogenic factor or a combination thereof.

In another embodiment, the one or more therapeutic agents administered with the active vitamin D compound or the mimic thereof may be actinomycin D, irinotecan, vincristine, vinblastine, vinorelbine, SN-38, azacitidine (5-azacytidine, 5AzaC), thalidomide, methotrexate, azathioprine, fluorouracil, doxorubicin, mitomycin, nitrates, calcium channel blockers, heparin, aspirin, coumarin, bishydroxycoumarin, warfarin, acid citrate dextrose, lepirudin, ticlopidine, clopidogrel, tirofiban, argatroban, and eptifibatide, blockers of IIb/IIIa receptors, hirudin, iloprost, sirolimus, everolimus, A24, tranilast, dexamethasone, tacrolimus, halofuginone, propyl hydroxylase, C-proteinase inhibitor, metalloproteinase inhibitor, corticosteroids, non-steroidal anti-inflammatory drugs, 17β-estradiol, angiotensin converting enzyme inhibitors, colchicine, fibroblast growth factor antagonists, histamine antagonists, lovastatin, nitroprusside, phosphodiesterase inhibitors, prostaglandin inhibitors, suramin, serotonin blockers, thioprotease inhibitors, platelet-derived growth factor antagonists, nitric oxide, or angiopeptin. In another embodiment, the one or more therapeutic agents administered with the active vitamin D compound or the mimic thereof may be anti-angiogenic factors such as bevacizumab, antineoplastic agents such as taxanes, vasodilators, anticoagulants, anti-platelet agents, anti-thrombins, immunosuppressants, anti-inflammatories, and collagen synthetase inhibitors. Examples of taxanes useful in this invention include paclitaxel and docetaxel. In one aspect of the invention, administration of vitamin D or a mimic thereof can start prior to administration of the one or more therapeutic agents and/or continue during and beyond administration of the one or more therapeutic agents. In another aspect of the invention, the method of administering an active vitamin D compound, or a mimic thereof, in combination with one or more therapeutic agents is repeated more than once.

A yet another aspect of the invention is directed towards a method of preventing, treating or ameliorating a thrombotic disorder in a human or non-human animal comprising administering to the animal a pharmaceutical composition comprising an effective amount of active vitamin D compound or a mimic thereof. In one aspect, the thrombotic disorder may be venous or arterial thrombosis, congestive heart failure, transient ischemic attacks, stroke, pulmonary embolism, arterial embolism, atherosclerosis, myocardial ischemia, myocardial infarction, cerebral thrombosis and ischemia, atherosclerosis and arteriosclerosis, angina, peripheral vascular disease, preeclampsia, or restenosis following angioplasty, carotid endarterectomy or anastomosis of vascular grafts. In another aspect, the active vitamin D compound is administered as a unit dosage form comprising about 10 μg to about 75 μg of calcitriol, about 50% MIGLYOL 812 and about 50% tocopherol PEG-1000 succinate (vitamin E TPGS). More preferably, the active vitamin D compound, or the mimic thereof, is administered as a unit dosage form comprising about 45 μg. In preferred embodiments of the invention, a combination of therapeutic agents is administered. In one embodiment of the invention, administration of vitamin D or a mimic thereof can start prior to administration of the one or more therapeutic agents and/or continue during and beyond administration of the one or more therapeutic agents. In another embodiment of the invention, the method of administering an active vitamin D compound, or a mimic thereof, in combination with one or more therapeutic agents is repeated more than once.

The combination of an active vitamin D compound, or a mimic thereof, with one or more therapeutic agents of the present invention can have additive potency or an additive therapeutic effect. The invention also encompasses synergistic combinations where the therapeutic efficacy is expected to be greater than additive. Preferably, such combinations will also reduce or avoid unwanted or adverse effects. In certain embodiments, the combination therapies encompassed by the invention are expected to provide an improved overall therapy relative to administration of an active vitamin D compound or a mimic thereof, or any therapeutic agent alone. In certain embodiments, doses of existing or experimental therapeutic agents can be reduced or administered less frequently which increases patient compliance, thereby improving therapy and reducing unwanted or adverse effects.

Further, the methods of the invention are useful not only with previously untreated patients but also useful in the treatment of patients partially or completely refractory to current standard and/or experimental therapies for prevention, treatment, or amelioration of thrombotic disorders. In a preferred embodiment, the invention provides therapeutic methods for the prevention, treatment, or amelioration of thrombotic disorders that has been shown to be or may be refractory or non-responsive to other therapies.

### DETAILED DESCRIPTION OF THE INVENTION

The invention involves the surprising discovery that late stage prostate cancer patients (i.e., patients with androgen independent prostate cancer) treated with Taxotere® and intermittent high doses of calcitriol experienced fewer serious cardiovascular adverse events as compared to patients receiving placebo or Taxotere® alone.

In one aspect of the invention, the active vitamin D compound, or the mimic thereof, is administered to an animal such that deep vein thrombosis or thrombophlebits is prevented, treated or ameliorated.

In another aspect of the invention, the active vitamin D compound, or the mimic thereof, has a reduced hypercalcemic effect, allowing higher doses of the compound to be administered to an animal without inducing severe symptomatic hypercalcemia.

A further aspect of the present invention is a method for preventing, treating, or ameliorating deep vein thrombosis or thrombophlebits in an animal comprising administering to the animal an active vitamin D compound, or a mimic thereof, by HDPA so that high doses of the active vitamin D compound, or the mimic thereof, can be administered to an animal without inducing severe symptomatic hypercalcemia.

In another aspect of the present invention, the active vitamin D compound or the mimic thereof is administered to an animal to prevent, treat or ameliorate thrombotic disorders. Thrombotic disorders include, but are not limited to, congestive heart failure, transient ischemic attacks, stroke, pulmonary embolism, arterial embolism, atherosclerosis, myocardial ischemia, myocardial infarction, cerebral thrombosis and ischemia, atherosclerosis and arteriosclerosis, angina, peripheral vascular disease, preeclampsia, or restenosis following angioplasty, carotid endarterectomy or anastomosis of vascular grafts. Thus, one aspect of the present invention is a method for preventing, treating, or ameliorating a cerebrovascular event (such as stroke) in an animal comprising administering to the animal an active vitamin D compound or a mimic thereof. Another aspect of the invention is a method for preventing, treating, or ameliorating myocardial infraction or ischemia by administering to an animal in need of such treatment an active vitamin D compound or a mimic thereof.

As used herein, the term “therapeutically effective amount” refers to that amount of the therapeutic agent sufficient to result in prevention of thrombosis, amelioration of one or more symptoms of thrombosis, or prevention of advancement of thrombosis. For example, with respect to the treatment of thrombosis or thrombotic disorders, a therapeutically effective amount preferably refers to the amount of a therapeutic agent that reduces the extent of thrombosis or a thrombotic disorder by at least 10%, preferably at least 20%, at least 30%, at least 40%, at least 50%, at least 60%, at least 70%, at least 80%, at least 90%, or at least 100%. For instance, the extent of thrombosis can be determined by any method known in the art for visualizing blood flow, e.g., contrast angiography. The extent of a thrombotic disorder may similarly be determined by any method known in the art for measuring systemic blood flow in the affected organ.

The terms “prevent, preventing, and prevention,” as used herein, are intended to refer to a decrease in the occurrence of thrombosis. The prevention may be complete, e.g., the total absence of thrombosis. The prevention may also be partial, such that the amount of thrombosis is less than that which would have occurred without the present invention. For example, the extent of thrombosis using the methods of the present invention may be at least 10%, preferably at least 20%, at least 30%, at least 40%, at least 50%, at least 60%, at least 70%, at least 80%, at least 90%, or at least 100% less than the amount of thrombosis that would have occurred without the present invention.

The term “thrombosis,” as used herein, refers to any condition in which a thrombus or a blood clot develops in a blood vessel or in the heart.

The term “venous thrombosis,” as used herein, refers to thrombosis of a vein with or without prior inflammation of the vein. For example, thrombosis may occur after angioplasty. The reoccurrence of thrombosis at the site of the angioplasty causes restinosis. The risk of thrombosis is often very high immediately after angioplasty because of the resultant tissue trauma, which tends to trigger blood clotting. Venous thrombosis may also occur without such tissue trauma. For example, thrombosis may be associated with sluggish blood flow or with rapid coagulation of the blood. According to the present invention, active vitamin D compounds or mimics thereof may be administered to animals having increased risk for venous thrombosis in order to prevent, ameliorate or treat venous thrombosis.

The term “deep venous thrombosis,” as used herein, refers to a condition where there is a blood clot (or thrombus) in a deep vein (i.e., a vein that accompanies an artery). Deep venous thrombosis (“DVT”) mainly affects the veins in the lower leg and the thigh and may interfere with blood circulation in the area. The blood clot may break off and travel through the blood stream (embolize) and lodge in the brain, lungs, heart, or other area, causing severe damage to the affected organ. Enhanced risk of deep venous thrombosis occurs during prolonged sitting (such as on long plane or car trips), bedrest or immobilization, recent surgery or trauma (especially hip, knee or gynecological surgery), fractures, childbirth within the last 6 months and the use of medications such as estrogen and birth control pills. Enhanced risk is also associated with a history of polycythemia vera, malignant tumor, and inherited or acquired hypercoagulability (changes in the levels of blood clotting factors making the blood more likely to clot). Although deep venous thrombosis is more commonly seen in adults over age 60, it can strike at any age. According to the present invention, active vitamin D compounds or mimics thereof may be administered to animals having increased risk for DVT in order to prevent, ameliorate or treat DVT.

The term “mesenteric venous thrombosis,” as used herein, refers to venous thrombosis of the mesenteric veins, which are the major veins that drain blood from the intestine. Mesenteric venous thrombosis compromises the blood supply to the intestine and can result in intestinal gangrene and tissue death.

The term “thrombotic disorder,” as used herein, refers to any disease condition that is a consequence of thrombo-embolic events of arterial and venous vasculature or thrombus formation in a blood vessel or in the heart. As used herein, thrombotic disorders include, but are not limited to, venous and arterial thrombosis, coronary insufficiency, heart disease, congestive heart failure, transient ischemic attacks, cerebrovascular accidents (e.g., stroke), pulmonary embolism, arterial embolism, atherosclerosis, myocardial ischemia, myocardial infarction, cerebral thrombosis and ischemia, atherosclerosis and arteriosclerosis, angina, peripheral vascular disease, preeclampsia, and restenosis following angioplasty, carotid endarterectomy or anastomosis of vascular grafts. Arterial embolism can affect the extremities—especially the legs and feet. It may involve the brain, causing a stroke, or the heart, causing a heart attack. Less common sites of arterial embolism include the kidneys, gut (intestines), and the eyes. Administering an active vitamin D compound, or a mimic thereof, to a human or a non-human animal decreases the risk of developing these thrombotic disorders.

Therapeutic agents useful as adjunctive therapy according to the invention include, but are not limited to, small molecules, synthetic drugs, peptides, polypeptides, proteins, nucleic acids (e.g., DNA and RNA polynucleotides including, but not limited to, antisense nucleotide sequences, triple helices, and nucleotide sequences encoding biologically active proteins, polypeptides, or peptides), antibodies, synthetic or natural inorganic molecules, mimetic agents, and synthetic or natural organic molecules. Any agent which is known to be useful, or which has been used or is currently being used for the prevention, treatment, or amelioration of thrombosis can be used in combination with an active vitamin D compound or the mimic thereof in accordance with the invention described herein

Therapeutic agents useful in the methods and compositions of the invention include antineoplastic agents (e.g., actinomycin D, irinotecan, vincristine, vinorelbine, SN-38, azacitidine (5-azacytidine, 5AzaC), thalidomide vinblastine, methotrexate, azathioprine, fluorouracil, doxorubicin, mitomycin, docetaxel, paclitaxel), anti-angiogenic factors, vasodilators (e.g., nitrates, calcium channel blockers), anticoagulants (e.g., heparin), anti-platelet agents (e.g., aspirin, blockers of IIb/IIIa receptors, clopidogrel), anti-thrombins (e.g., hirudin, iloprost), immunosuppressants (e.g., sirolimus, tranilast, dexamethasone, tacrolimus, everolimus, A24), collagen synthetase inhibitors (e.g., halofuginone, propyl hydroxylase, C-proteinase inhibitor, metalloproteinase inhibitor), anti-inflammatories (e.g., corticosteroids, non-steroidal anti-inflammatory drugs), 17β-estradiol, angiotensin converting enzyme inhibitors, colchicine, fibroblast growth factor antagonists, histamine antagonists, lovastatin, nitroprusside, phosphodiesterase inhibitors, prostaglandin inhibitors, suramin, serotonin blockers, thioprotease inhibitors, platelet-derived growth factor antagonists, nitric oxide, and angiopeptin. In one embodiment, the therapeutic agent is a taxane, e.g., paclitaxel or docetaxel.

In some embodiments, the active vitamin D compound or the mimic thereof is administered in combination with agents, such as anti-angiogenic agents, that block, inhibit or modulate tumor neovascularization. In preferred embodiments, anti-angiogenesis agents can be any anti-angiogenesis agent which is used, has been used, or is known to be useful for the treatment of hyperproliferative disorders. Examples of anti-angiogenesis agents include bevacizumab (AVASTIN®), VEGF-TRAP, anti-VEGF-receptor antibodies, angiostatin, endostatin, batimastat, captopril, cartilage derived inhibitor, genistein, interleukin 12, lavendustin, medroxypregesterone acetate, recombinant human platelet factor 4, tecogalan, thrombospondin, TNP-470, VEGF antagonists, anti-VEGF monoclonal antibody, soluble VEGF-receptor chimaeric protein, antisense oligonucleotides, antisense oligodexoynucleotides, siRNAs, anti-VEGF aptamers, pigment epithelium derived factor, a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, pentosan polysulfate, a cyclooxygenase inhibitor, carboxyamidotriazole, combretastatin A-4, squalamine, 6-O-chloroacetyl-carbonyl)-fumagillol, thalidomide, troponin-1, indolinethiones, pyridopyrimidines, quinoazolines, phenyl-pyrrolo-pyrimidines, trastuzumab, calcium influx inhibitor (CAI), neomycin, squalamine, marimastat, prinomastat (AG-3340), metastat (COL-3) and cinnoline derivatives. Additional anti-angiogenic compounds that may be administered in combination with the compounds of the present invention are described in U.S. Pat. Nos. 5,192,744, 5,426,100, 5,733,876, 5,840,692, 5,854,205, 5,990,280, 5,994,292, 6,342,219, 6,342,221, 6,346,510, 6,479,512, 6,719,540, 6,797,488, 6,849,599, 6,869,952, 6,887,874, 6,958,340 and 6,979,682.

Administration of some anti-angiogenic factors to patients in need of such treatment is known to cause serious adverse events. For example, various serious adverse events are known to be associated with the administration of AVASTIN® to patients, including gastrointestinal perforation, hemorrhage, arterial thromboembolic events, hypertensive crisis, nephrotic syndrome and congestive heart failure, in a trial in patients with untreated metastatic colorectal cancer. See AVASTIN® Product Label, Genentech, Inc.

In one study evaluating AVASTIN® as first line treatment of metastatic carcinoma of the colon or rectum (referred to as Study 1 in the AVASTIN® product label), 18% of patients receiving bolus-IFL (irinotecan, 5-flourouracil and leucovorin) plus AVASTIN® and 15% of patients receiving bolus-IFL plus placebo experienced a Grade 3-4 thromboembolic event. The incidence of the following Grade 3 and 4 thromboembolic events was higher in patients receiving bolus-IFL plus AVASTIN® as compared to patients receiving bolus-IFL plus placebo: cerebrovascular events (4 vs. 0 patients), myocardial infraction (6 vs. 3), deep venous thrombosis (34 vs. 19) and intra-abdominal thrombosis (13 vs. 5). The incidence of pulmonary embolism was higher in patients receiving bolus-IFL plus placebo (16 vs. 20 patients). Moreover, 53 of 392 (14%) patients who received bolus-IFL plus AVASTIN® and 30 of 396 (8%) patients who received bolus-IFL plus placebo had a thromboembolic event and received full-dose warfarin. Two patients in each treatment arm (four total) developed bleeding complications. Eleven of 53 (21%) patients receiving bolus-IFL plus AVASTIN® and one of 30 (3%) patients receiving bolus-IFL developed an additional thromboembolic event. See AVASTIN® Product Label, Genentech, Inc.

To ameliorate, prevent or treat these thrombotic disorders and other side effects associated with the administration of AVASTIN®, the active vitamin D compound or the mimic thereof may be administered in combination with AVASTIN®. The vitamin D compound or mimic thereof may be administered prior to the administration of AVASTIN® (e.g., 1-3 days prior to administration of AVASTIN®, concurrent with the administration of AVASTIN® and/or after administration of AVASTIN®. In some embodiments, the active vitamin D compound or the mimic thereof, AVASTIN® and one or more therapeutic agents may be administered. In further embodiments, the one or more therapeutic agents may be chemotherapeutic agents such as alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, angiogenesis inhibitors and other anti-angiogenic agents.

Chemotherapeutic agents that may be combined with the active vitamin D compound or the mimic thereof and AVASTIN® include, but are not limited to, abarelix, aldesleukin, alemtuzumab, alitretinoin, allopurinol, altretamine, amifostine, anastrozole, arsenic trioxide, asparaginase, BCG live, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, 5-flourouracil, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

Anti-inflammatory drugs suitable for ameliorating inflammations associated with pulmonary disorders include salicylates (such as aspirin, choline magnessium trisalicylate, methyl salicylate, salsalte and diflunisal), acetic acids (such as indomethacin, sulindac, tolmetin, aceclofenac and diclofenac), 2-arylpropionic acids or profens (such as ibuprofen, ketoprofen, naproxen, fenoprofen, flurbiprofen and oxaprozin), N-arylanthranilic acids or fenamic acids (such as mefenamic acid, flufenamic acid, and meclofenamate), enolic acids or oxicams (such as piroxicam and meloxicam), cox inhibitors (such as celecoxib, rofecoxib (withdrawn from market), valdecoxib, parecoxib and etoricoxib), sulphonanilides such as nimesulide; naphthylalkanones (such as nabumetone), pyranocarboxylic acids (such as etodolac) and pyrroles (such as ketorolac).

As used herein, the term “immunomodulatory agent” and variations thereof including, but not limited to, immunomodulatory agents, immunomodulants, immunomodulators or immunomodulatory drugs, refer to an agent that modulates a host's immune system. In particular, an immunomodulatory agent is an agent that alters the ability of a subject's immune system to respond to one or more foreign antigens. In a specific embodiment, an immunomodulatory agent is an agent that shifts one aspect of a subject's immune response, e.g., the agent shifts the immune response from a Th1 to a Th2 response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent).

Immunomodulatory agents useful for the present invention include, but are not limited to, small molecules, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. A particularly useful immunomodulatory agent useful for the present invention is thalidomide.

Immunosuppressant agents are useful to counteract autoimmune diseases, such as rheumatoid arthritis or Crohn's disease, and to prevent the immune system from attacking healthy parts of the body. In some embodiments, immunosuppressive agents useful for the present invention include glucocorticoid receptor agonists (e.g., cortisone, dexamethasone, hydrocortisone, betamethasone), calcineurin inhibitors (e.g., macrolides such as tacrolimus and pimecrolimus), immunophilins (e.g., cyclosporin A) and mTOR inhibitors (e.g., sirolimus, marketed as RAPAMUNE® by Wyeth). In other embodiments, immunomodulatory agents useful for the present invention further include antiproliferative agents (e.g., methotrexate, leflunomide, cisplatin, ifosfamide, paclitaxol, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, taxol, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, melphalan, glucocorticoids, procaine, tetracaine, lidocaine, propranolol, puromycin homologs, and cytoxan.

Immunostimulant agents are useful to increase the efficiency of the immune system and treat immunodeficiency disorders. Immunostimulant agents useful for the present invention include interferon and Zidovudine (AZT).

The term “an active vitamin D compound or a mimic thereof in combination with one or more therapeutic agents,” as used herein, is intended to refer to the combined administration of an active vitamin D compound or a mimic thereof and one or more therapeutic agents, wherein the active vitamin D compound or the mimic thereof can be administered prior to, concurrently with, or after the administration of the therapeutic agents. The active vitamin D compound or the mimic thereof can be administered up to three months prior to or after the therapeutic agents and still be considered to be a combination treatment.

The term “active vitamin D compound,” as used herein, is intended to refer to a vitamin D compound that is or becomes biologically active when administered to a subject or contacted with cells. The biological activity of a vitamin D compound can be assessed by assays well known to one of skill in the art such as, e.g., immunoassays that measure the expression of a gene regulated by vitamin D. Vitamin D compounds exist in several forms with different levels of activity in the body. For example, a vitamin D compound may be partially activated by first undergoing hydroxylation in the liver at the carbon-25 position and then may be fully activated in the kidney by further hydroxylation at the carbon-1 position. The prototypical active vitamin D compound is 1α,25-hydroxyvitamin D₃, also known as calcitriol. The active vitamin D compound of the present invention may also be a partially hydroxylated vitamin D such as 1α-hydroxyvitamin D₃, also known as 1α-calcidol, and 25-hydroxyvitamin D₃, also known as calcifediol. A large number of other active vitamin D compounds are known and can be used in the practice of the invention. The active vitamin D compounds of the present invention include, but are not limited to, the analogs, homologs and derivatives of vitamin D compounds described in the following patents, each of which is incorporated by reference: U.S. Pat. No. 4,391,802 (1α-hydroxyvitamin D derivatives); U.S. Pat. No. 4,717,721 (1α-hydroxy derivatives with a 17 side chain greater in length than the cholesterol or ergosterol side chains); U.S. Pat. No. 4,851,401 (cyclopentano-vitamin D analogs); U.S. Pat. Nos. 4,866,048 and 5,145,846 (vitamin D₃ analogues with alkynyl, alkenyl, and alkanyl side chains); U.S. Pat. No. 5,120,722 (trihydroxycalciferol); U.S. Pat. No. 5,547,947 (fluoro-cholecalciferol compounds); U.S. Pat. No. 5,446,035 (methyl substituted vitamin D); U.S. Pat. No. 5,411,949 (23-oxa-derivatives); U.S. Pat. No. 5,237,110 (19-nor-vitamin D compounds; U.S. Pat. No. 4,857,518 (hydroxylated 24-homo-vitamin D derivatives). Particular examples include ROCALTROL (Roche Laboratories); CALCIJEX injectable calcitriol; investigational drugs from Leo Pharmaceuticals including EB 1089 (24a,26a,27a-trihomo-22,24-diene-1αa,25-(OH)₂-D₃, KH 1060 (20-epi-22-oxa-24a,26a,27a-trihomo-1α,25-(OH)₂-D₃), MC 1288 (1,25-(OH)₂-20-epi-D₃) and MC 903 (calcipotriol, 1α24s-(OH)₂-22-ene-26,27-dehydro-D₃); Roche Pharmaceutical drugs that include 1,25-(OH)₂-16-ene-D₃, 1,25-(OH)₂-16-ene-23-yne-D₃, and 25-(OH)₂-16-ene-23-yne-D₃; Chugai Pharmaceuticals 22-oxacalcitriol (22-oxa-1α,25-(OH)₂-D₃; 1α-(OH)-D₅ from the University of Illinois; and drugs from the Institute of Medical Chemistry-Schering AG that include ZK 161422 (20-methyl-1,25-(OH)₂-D₃) and ZK 157202 (20-methyl-23-ene-1,25-(OH)₂-D₃); 1α-(OH)-D₂; 1α-(OH)-D₃ and 1α-(OH)-D₄. Additional examples include 1α,25-(OH)₂-26,27-d₆-D₃; 1α,25-(OH)₂-22-ene-D₃; 1α,25-(OH)₂-D₃; 1α,25-(OH)₂-D₂; 1α,25-(OH)₂-D₄; 1α,24,25-(OH)₃-D₃; 1α,24,25-(OH)₃-D₂; 1α,24,25-(OH)₃-D₄; 1α-(OH)-25-FD₃; 1α-(OH)-25-FD₄; 1α-(OH)-25-FD₂; 1α,24-(OH)₂-D₄; 1,24-(OH)₂-D₃; 1α,24-(OH)₂-D₂; 1α,24-(OH)₂-25-FD₄; 1α,24-(OH)₂-25-FD₃; 1α,24-(OH)₂-25-FD₂; 1α,25-(OH)₂-26,27-F₆-22-ene-D₃; 1α,25-(OH)₂-26,27-F6-D₃; 1α,25S—(OH)₂-26-F₃-D₃; 1α,25-(OH)₂-24-F₂-D₃; 1α,25S,26-(OH)₂-22-ene-D₃; 1α,25R, 26-(OH)₂-22-ene-D₃; 1α,25-(OH)₂-D₂; 1α,25-(OH)₂-24-epi-D₃; 1α,25-(OH)₂-23-yne-D₃; 1α,25-(OH)₂-24R—F-D₃; 1α,25S,26-(OH)₂-D₃; 1α,24R—(OH)₂-25F-D₃; 1α,25-(OH)₂-26,27-F₆-23-yne-D₃; 1α,25R—(OH)₂-26-F3-D₃; 1α,25,28-(OH)₃-D₂; 1α,25-(OH)₂-16-ene-23-yne-D₃; 1α,24R,25-(OH)₃-D₃; 1α,25-(OH)₂-26,27-F₆-23-ene-D₃; 1α,25R—(OH)₂-22-ene-26-F₃-D₃; 1α,25S—(OH)₂-22-ene-26-F₃-D₃; 1α,25R—(OH)₂-D₃-26,26,26-d₃; 1α,25S—(OH)₂-D₃-26,26,26-d₃; and 1α,25R—(OH)₂-22-ene-D₃-26,26,26-d₃. Additional examples can be found in U.S. Pat. No. 6,521,608. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791.

The term “mimic” as used herein is intended to refer to non-secosteroidal vitamin D mimic compounds. In general, these non-secosteroidal vitamin D mimics are compounds that do not structurally fall within the class of compounds generally known as vitamin D compounds but which modulate the activity of vitamin D nuclear receptors. Examples of such vitamin D mimics include bis-aryl derivatives disclosed by U.S. Pat. No. 6,218,430 and WO publication 2005/037755. Additional examples of non-secosteroidal vitamin D mimic compounds suitable for the present invention can be found in U.S. Pat. Nos. 6,831,106; 6,706,725; 6,689,922; 6,548,715; 6,288,249; 6,184,422, 6,017,907, 6,858,595 and 6,358,939.

In one aspect the invention is drawn to methods employing non-secosteroidal vitamin D mimic compounds having Formula I:

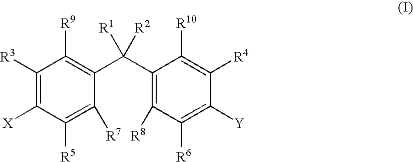

wherein:

R¹ and R² are each independently halo, haloalkyl, pseudohalo, optionally substituted alkyl, optionally substituted alkenyl, optionally substituted alkynyl, optionally substituted cycloalkyl, optionally substituted heterocyclyl, optionally substituted aryl or optionally substituted heteroaryl; or

R¹ and R², together with the carbon atom to which they are attached, form an optionally substituted cycloalkyl consisting of:

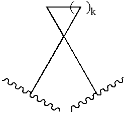

wherein k is an integer from 1 to 6; or

R¹ and R², together with the carbon atom to which they are attached, form an optionally substituted heterocyclyl selected from a group consisting of: 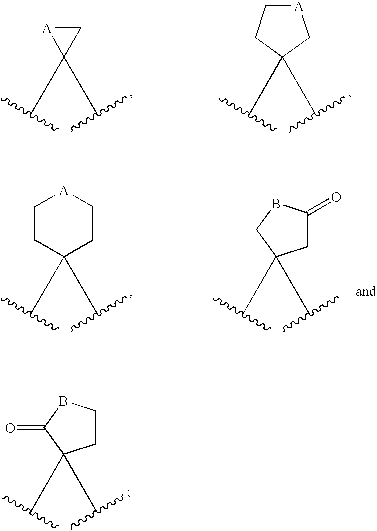
wherein A is —O—, —NR^{x}—, —S—, —S(O)— or —S(O)₂— wherein RX is hydrogen, alkyl, haloalkyl, cycloalkyl, heterocyclyl, aryl, heteroaryl, —R¹⁴—C(J)R¹⁵, —R¹⁴—C(J)OR¹⁵, —R¹⁴—C(J)R¹⁶OR¹⁵, —R¹⁴—C(J)SR¹⁶, —R¹⁴—C(J)N(R¹⁸)R¹⁹, —R¹⁴—C(J)N(R¹⁷)N(R¹⁸)R¹⁹, —R¹⁴—C(J)N(R¹⁷)S(O)ₚR²⁰, —R¹⁴—S(O)ₚN(R¹⁸)R¹⁹, or —R¹⁴—S(O)ₚR²⁰; and wherein B is —O—, —S— or —NR^{y} where R^{y} is hydrogen, alkyl, haloalkyl, aryl or heteroaryl; and wherein each p is independently 0 to 2;

R³ and R⁴ are each independently hydrogen, alkyl, alkenyl, alkynyl, cycloalkyl, heterocyclyl, aryl, heteroaryl, halo, pseudohalo, nitro, cyano, azido, —R¹⁴—OR¹⁵, —R¹⁴—N(R¹⁸)R¹⁹, —R¹⁴—SR¹⁵, —R¹⁴—OC(J)R¹⁵, —R¹⁴—NR¹⁷C(J)R¹⁵, —R¹⁴—OC(J)N(R¹⁸)R¹⁹, —R¹⁴—NR¹⁷C(J)N(R¹⁸)R¹⁹, —R¹⁴—NR¹⁷C(J)OR¹⁵, —R¹⁴—C(J)R¹⁵, —R¹⁴—C(J)OR¹⁵, —R¹⁴—C(J)SR¹⁵, —R¹⁴—C(J)N(R¹⁸)R¹⁹, or —R¹⁴—C(J)N(R¹⁷)N(R¹⁸)R¹⁹;

R⁵, R⁶, R⁷, R⁸, R⁹, R¹⁰ are each independently hydrogen, halo, hydroxy, amino, pseudohalo, cyano, nitro, alkyl, haloalkyl, alkoxy or haloalkoxy;

X is R²⁵;

Y is independently R³⁰, —OR³¹, —SR³² or —N(R³³)(R³⁴);

R²⁵ and R³⁰ are each independently selected from (i) or (ii) as follows:

(i) optionally substituted alkyl that may be substituted with one to ten substituents each independently selected from a group consisting of halo, pseudohalo, nitro, cyano, thioxo, azido, amidino, guanidino, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)(R¹⁸)R¹⁹, —C(J)N(R¹⁷)N(R¹⁸)R¹⁹, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹ and —C(═NR¹⁷)N(R¹⁸)R¹⁹; or

(ii) optionally substituted alkenyl or optionally substituted alkynyl, either of which may be substituted with one to ten substituents each independently selected from a group consisting of oxo, thioxo, halo, pseudohalo, nitro, cyano, azido, amidino, guanidino, —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —S(O)ₚR²⁰, —N(R¹⁷)S(O)ₚR²⁰, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, haloalkyl, cycloalkyl, heterocyclyl, aryl and heteroaryl;

R³¹, R³², R³³, and R³⁴ are each independently optionally substituted alkyl, optionally substituted alkenyl, optionally substituted alkynyl or optionally substituted cycloalkyl; all of which may be optionally substituted with one to ten substituents each independently selected from a group consisting of oxo, halo, pseudohalo, nitro cyano, azido, amidino, guanidino —OR¹⁵, —OR¹⁶OR¹⁵, —N(R¹⁸)R¹⁹, —N(R¹⁷)N(R¹⁸)R¹⁹, —SR¹⁵, —SR¹⁶SR¹⁵, —S(O)ₚR²⁰, —N(R¹⁷)S(O)ₚR²⁰, —N(R¹⁷)N(R¹⁷)S(O)ₚR²⁰, —OC(J)R¹⁵, —NR¹⁷C(J)R¹⁵, —OC(J)N(R¹⁸)R¹⁹, —NR¹⁷C(J)N(R¹⁸)R¹⁹, —NR¹⁷C(J)OR¹⁵, —OC(J)OR¹⁵, —P(R²¹)₂, —P(O)(R²¹)₂, —OP(O)(R²¹)₂, —C(J)R¹⁵, —C(J)OR¹⁵, —C(J)SR¹⁶, —C(J)N(R¹⁸)R¹⁹, —C(J)N(R¹⁷)N(R¹⁸)R¹⁹, —C(J)N(R¹⁷)S(O)ₚR²⁰, —C(J)N(R¹⁷)N(R¹⁷)S(O)ₚR²⁰, —C(R¹⁷)═NOR¹⁵, —C(R¹⁷)═NR¹⁷, —C(R¹⁷)═NN(R¹⁸)R¹⁹, —C(═NR¹⁷)N(R¹⁸)R¹⁹, alkyl, cycloalkyl, heterocyclyl, aryl and heteroaryl, and R³⁴ can additionally be hydrogen;

where each R¹⁴ is independently a direct bond or alkylene;

where each R¹⁵ and R¹⁷ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, cyano, hydroxy and amino;

where each R¹⁶ and R²⁰ is independently optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino; and

where each R¹⁸ and R¹⁹ is independently hydrogen, optionally substituted alkyl, optionally substituted alkenyl, optionally substituted alkynyl, optionally substituted cycloalkyl, optionally substituted heterocyclyl, optionally substituted aryl or optionally substituted heteroaryl, all of which, when substituted, are substituted with one to five substituents each independently selected from halo, hydroxy, alkoxy and amino;

or where R¹⁸ and R¹⁹, together with the nitrogen atom to which they are attached, form a heterocyclyl or heteroaryl;

each R²¹ is independently alkyl, —OR²² or —N(R²³)R²⁴;

R²² is hydrogen, alkyl, haloalkyl, alkenyl, alkynyl, cycloalkyl, heterocyclyl, aryl, heteroaryl or aralkyl;

R²³ and R²⁴ are each independently hydrogen, alkyl, haloalkyl, alkenyl, alkynyl or cycloalkyl;

or R²³ and R²⁴, together with the nitrogen atom to which they are attached, form a heterocyclyl or heteroaryl;

each J is independently O or S;

as a single isomer, a mixture of isomers, or as a racemic mixture of isomers; as a solvate or polymorph; or as a prodrug or metabolite; or as a pharmaceutically acceptable salt thereof.

In one embodiment, R¹ and R² may form a substituted cyclohexyl, said cyclohexyl, when substituted at the 4-position relative to the gem-diaryl substituents, may be substituted with a substituent selected from the group consisting of halo, cyano, optionally substituted alkyl, optionally substituted alkenyl, optionally substituted alkynyl, optionally substituted aryl and optionally substituted heteroaryl.

In another embodiment, R²⁵ and R³⁰ are not —CH₂COOH; —CH₂-5-tetrazolyl; —CH₂COOMe; —CH₂COOEt; —CH₂NH(CH₂COOH); —CH₂N(C(O)Me)(CH₂COOH); —CH₂—N-pyrrolidin-2-one; —CH₂-(1-methylpyrrolidin-2-one-3-yl); —CH₂C(O)NH₂; —CH₂C(O)NMe₂; —CH₂C(O)NHMe; —CH₂C(O)—N-pyrrolidone; —CH(OH)COOH; —CH(OH)C(O)NH₂; —CH(OH)C(O)NHMe; —CH(OH)C(O)NMe₂; —CH(OH)C(O)NEt₂; —CH₂CH₂COOH; —CH₂CH₂COOMe; —CH₂CH₂COOEt; —CH₂CH₂C(O)NH₂; —CH₂CH₂C(O)NHMe; —CH₂CH₂C(O)NMe₂; or —CH₂CH₂-5-tetrazolyl.

In another aspect the invention is drawn to methods employing the following non-secosteroidal vitamin D mimic compounds:
3-(2-methyl-4-{2,2,2-trifluoro-1-[4-(2-hydroxy-3,3-dimethyl-butoxy)-3-methyl-phenyl]-1-phenyl-ethyl}-phenoxy)-propane-1,2-diol;
3-(4-{4-[4-(2-hydroxy-3,3-dimethyl-butoxy)-3-methyl-phenyl]-piperidin-4-yl}-2-methyl-phenoxy)-propane-1,2-diol;
3-(4-{4-[4-(2-hydroxy-3,3-dimethyl-butoxy)-3-methyl-phenyl]-piperidin-4-yl}-2-methyl-phenoxy)-propane-1,2(S)-diol;
1-{4-[4-(2(S),3-dihydroxy-propoxy)-3-methyl-phenyl]-4-[4-(2-hydroxy-3,3-dimethyl-butoxy)-3-methyl-phenyl]-piperidin-1-yl}-ethanone;
1-(4-{1-acetyl-4-[4-(3,3-dimethyl-2-oxo-butoxy)-3-methyl-phenyl]-piperidin-4-yl}-2-methyl-phenoxy)-3,3-dimethyl-butan-2-one;
3-(4-{1-ethyl-1-[4-(3-hydroxy-3-methylbutyl)-3-methylphenyl]-propyl}-2-methylphenoxy)-propane-1,2(S)-diol;
3-(4-{1-ethyl-1-[4-(3-ethyl-3-hydroxypentyl)-3-methylphenyl]-propyl}-2-methyl-phenoxy)-propane-1,2(S)-diol;
3-(4-{1-ethyl-1-[4-(3-hydroxy-5-methylhexyl)-3-methylphenyl]-propyl}-2-methyl-phenoxy)-propane-1,2(S)-diol;
3-(4-{1-ethyl-1-[4-(3-hydroxy-4-methylpentyl)-3-methylphenyl]-propyl}-2-methyl-phenoxy)-propane-1,2(S)-diol;
3-(2-ethyl-4-{1-ethyl-1-[4-(3-hydroxy-4,4-dimethylpentyl)-3-methylphenyl]-propyl}-phenoxy)-propane-1,2(S)-diol;
3-(4-{1-ethyl-1-[4-(3-hydroxy-4,4-dimethylpentyl)-3-methylphenyl]-propyl}-2-methyl-phenoxy)-propane-1,2(S)-diol;
3-[4-(1-ethyl-1-{4-[3(S)-hydroxy-4,4-dimethylpentyl]-3-methylphenyl}-propyl)-2-methyl-phenoxy]-propane-1,2(S)-diol;
3-[4-(1-ethyl-1-{4-[3(R)-hydroxy-4,4-dimethylpentyl]-3-methylphenyl}-propyl)-2-methyl-phenoxy]-propane-1,2(S)-diol and
3-(4-{1-ethyl-1-[4-(3-hydroxy-4,4-dimethylpentyl)-phenyl]-propyl}-2-methylphenoxy)-propane-1,2(S)-diol.

In another aspect the invention is drawn to methods employing non-secosteroidal vitamin D mimic compounds having Formula II:

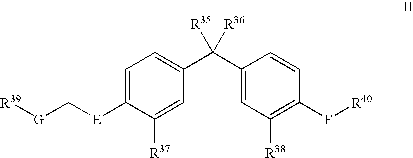

wherein:

E and F are each independently selected from the group consisting of O, S, and NR⁴¹;

G is selected from the group consisting of C═O, CH(OR⁴²), and CH(NR⁴³R⁴⁴);

R³⁵ and R³⁶ are independently selected from the group consisting of alkyl groups, optionally fluorinated; or together R³⁵ and R³⁶ form a cycloalkylidene having 3 to 8 carbon atoms, optionally fluorinated;

R³⁷ and R³⁸ are independently selected from the group consisting of halogen; lower n-alkyl, optionally fluorinated; and lower alkoxy, optionally fluorinated;

R³⁹ is selected from the group consisting of H; optionally substituted alkyl groups; optionally substituted alkenyl groups; optionally substituted alkynyl groups; optionally substituted aryl groups; OR⁴⁵; NR⁴⁶R⁴⁷; or together with R⁴², R⁴³, or R⁴⁴ forms a 3- to 12-membered cyclic group wherein said cyclic group is selected from the group consisting of amidines, amines, ethers, lactams, lactones, ketals, hemiketals, aminals, hemiaminals, carbonates, carbamates, ureas, and combinations thereof;

R⁴⁰ is selected from the group consisting of H and alkyl groups, optionally substituted;

R⁴¹ is selected from the group consisting of H and alkyl groups, optionally substituted;

R⁴² is selected from the group consisting of H, optionally substituted alkyl groups, optionally substituted alkenyl groups, optionally substituted alkynyl groups, optionally substituted aryl group, and optionally substituted acyl groups;

R⁴³ and R⁴⁴ are independently selected from the group consisting of H, optionally substituted alkyl groups, optionally substituted alkenyl groups, optionally substituted alkynyl groups, optionally substituted aryl groups, and optionally substituted acyl groups;

R⁴⁵ is selected from the group consisting of H, optionally substituted alkyl groups, optionally substituted alkenyl groups, optionally substituted alkynyl groups, optionally substituted aryl groups, and optionally substituted acyl groups; and

R⁴⁶ and R⁴⁷ are independently selected from the group consisting of H, optionally substituted alkyl groups, optionally substituted alkenyl groups, optionally substituted alkynyl groups, optionally substituted aryl groups, and optionally substituted acyl groups and pharmaceutically acceptable salts thereof.

In a first embodiment, when K and L are both O, M is C═O, and R⁴⁵ is selected from the group consisting of OH and C₁-C₄ alkoxy, then R⁴⁶ is not carboxymethyl and alkyl esters thereof. In a second embodiment, when K and L are both 0, and M is selected from the group consisting of CH(OR⁴⁸) and CH(NR⁴⁹R⁵⁰), then R⁴⁵ is not H or primary alkyl. In a third embodiment, when K and L are both 0, and M is CH(OR⁴⁸), then R⁴⁶ and R⁴⁸ do not both comprise aziridines. In a fourth embodiment, when K and L are both 0, and M is CH(OR⁴⁸), then R⁴⁵, R⁴⁶, and R⁴⁸ do not simultaneously comprise alkenyl ethers. In a fifth embodiment, when K and L are both 0, and M is CH(OR⁴⁸), then R⁴⁵ and R⁴⁶ do not both comprise glycidyl ethers.

The term “high dose pulse administration” (HDPA) as used herein is intended to refer to a regimen of administration of an active vitamin D compound or mimic thereof to an animal which achieves an antithrombotic effect in the animal without inducing severe symptomatic hypercalcaemia, e.g., a dose of at least 0.5 μg no more than once every three days.

The term “hypercalcemia” as used herein refers to a medical condition in which the concentration of calcium ions in the plasma is greater than about 10.5 mg/dL in humans.

The term “symptomatic hypercalcemia” as used herein refers to symptoms associated with one of more of the signs or symptoms of hypercalcemia. Early manifestations of hypercalcemia include weakness, headache, somnolence, nausea, vomiting, dry mouth, constipation, muscle pain, bone pain, or metallic taste. Late manifestations include polydypsia, polyuria, weight loss, pancreatitis, photophobia, pruritis, renal dysfunction, aminotransferase elevation, hypertension, cardiac arrhythmias, psychosis, stupor, or coma. Methods to determine the concentration of calcium ions in blood plasma are generally within the capability of a person of ordinary skill in the art.

The term “severe symptomatic hypercalcemia” as used herein is referred to grade 3 or grade 4 toxic level of hypercalcemia as defined in U.S. Pat. No. 6,521,608, which is incorporated by reference herein in its entirety. A grade 4 toxicity is associated with reduced count for WBC, platelets, hemoglobin, neutrophils and lymphocytes; massive hemorrhage; gastrointestinal problems (such as vomiting more than 10 times a day, diarrhea (>10 times a day) and stomatitis which requires IV nutrition); hepatic failures (such as elevated bilirubin and hepatic coma), kidney/bladder dysfunction; cardiovascular events (such as refractory congestive heart failure, acute myocardial infraction, dyspnea at rest and cardiac tamponade); neuralgic disorders (such as paralysis, coma, seizures, cerebellar necrosis, severe headaches, blindness, uncorrectable deafness and suicidal mood) and metabolic problems (such as hyperglycemia (blood glucose >500 mg/dL) with ketoacidosis). Although grade 3 toxicity is milder than grade 4 toxicity, it can be life threatening and is associated with reduced count for WBC, platelets, hemoglobin, neutrophils and lymphocytes; gross hemorrhage; gastrointestinal problems (such as vomiting 6-10 times a day, diarrhea (7-9 times a day) and painful ulcers (patient could not eat)); hepatic failures (such as precoma and elevated bilirubin); cardiovascular events (such as mild congestive heart failure responsive to treatment, angina without infraction and symptomatic effusion); neurologic disorders (such as severe loss or impairment of neuro-sensory, severe cortical contusion, unrelenting headache and correctable hearing loss) and weight change.

In a preferred embodiment of the invention, the active vitamin D compound or mimic thereof has a reduced hypercalcemic effect as compared to vitamin D so that increased doses of the compound can be administered without inducing hypercalcemia in the animal. A reduced hypercalcemic effect is defined as an effect which is less than the hypercalcemic effect induced by administration of an equal dose of 1α,25-hydroxyvitamin D₃ (calcitriol). As an example, EB 1089 has a hypercalcemic effect which is 50% of the hypercalcemic effect of calcitriol. Additional active vitamin D compounds having a reduced hypercalcemic effect include Ro23-7553 and Ro24-5531 available from Hoffmann LaRoche. Other examples of active vitamin D compounds having a reduced hypercalcemic effect can be found in U.S. Pat. No. 4,717,721. Determining the hypercalcemic effect of an active vitamin D compound is routine in the art and can be carried out as disclosed in Hansen et al., Curr. Pharm. Des. 6:803-828 (2000).

In one embodiment of the invention, an active vitamin D compound or a mimic thereof is administered to an animal before, during and/or after an angioplasty procedure or bypass procedure. The active vitamin D compound or the mimic thereof can be administered 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more prior to the angioplasty or bypass procedure. The active vitamin D compound or the mimic thereof can be administered 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more after the angioplasty or bypass procedure and continued for up to six months. In certain embodiments the active vitamin D compound or the mimic thereof is administered before, during, and after the angioplasty procedure or bypass procedure.

In one aspect of the invention, one or more therapeutic agents are administered to an animal in addition to the active vitamin D compound or the mimic thereof. The active vitamin D compound or the mimic thereof can be administered prior to (e.g., 0.5 hours, 1 hour, 2 hours, 4 hours, 6 hours, 12 hours, 24 hours, 36 hours, 2 days, 3 days, 4 days, 5 days, 6 days, 7 days, 2 weeks, 3 weeks, 4 weeks or more), concurrently with, or after (e.g., 0.5 hours, 1 hour, 2 hours, 4 hours, 6 hours, 12 hours, 24 hours, 36 hours, 2 days, 3 days, 4 days, 5 days, 6 days, 7 days, 2 weeks, 3 weeks, 4 weeks or more) the administration of one or more therapeutic agents.

In certain embodiments, the method of administering an active vitamin D compound, or a mimic thereof, in combination with one or more therapeutic agents may be repeated at least once. The method may be repeated as many times as necessary to achieve or maintain a therapeutic response, e.g., from one to about ten times. With each repetition of the method the active vitamin D compound, or the mimic thereof, and the one or more therapeutic agents may be the same or different from that used in the previous repetition. Additionally, the time period of administration of the active vitamin D compound and the manner in which it is administered (i.e., daily or HDPA) can vary from repetition to repetition.

When used, the one or more therapeutic agents are administered in doses known to one of skill in the art to prevent, treat, or ameliorate thrombosis. The one or more therapeutic agents are administered in pharmaceutical compositions and by methods known to be effective. For example, the therapeutic agents may be administered systemically (e.g., intravenously, orally) or locally.

The doses of the vitamin D analogs and vitamin D mimics may be adjusted proportionate to the ratio of the efficacy index to the calcemic index according to the formula:
Dose=CalcitriolDose×(EI÷CI)

where Dose is the analog or mimic dose, calcitriolDose is calcitriol dose, EI is the analog or mimic efficacy index and CI is the analog or mimic calcemic index, wherein the term “efficacy index” is the ratio of the concentration of the vitamin D analog or mimic to the concentration of calcitriol at equivalent potency. Thus, the efficacy index is a fraction less than one when the vitamin D analog or mimic is less potent than calcitriol. EI is number greater than one when calcitriol is less potent than the vitamin D analog or mimic. The “calcemic index” of a drug is a measure of the relative ability of the drug to generate a calcemic response as reported in Bouillon et al., Endocrine Reviews 16:200-257, 1995. A calcemic index of 1 corresponds to the relative calcemic activity of calcitriol. A calcemic index of about 0.01 corresponds to the calcemic activity of a drug with approximately 100 times less calcemic activity than calcitriol. A calcemic index of 0.5 would correspond to a drug having approximately half the calcemic activity of calcitriol. The calcemic index of a drug can vary depending on the assay conducted, e.g. whether one is measuring stimulation of intestinal calcium absorption (a process by which dietary calcium enters into the physiological processes to contribute to the skeletal growth of the organism and to the maintenance of calcium homeostasis) or bone calcium mobilizing activity (a process by which the bone matrix acts as an exchangeable reservoir for calcium). See U.S. Pat. No. 6,521,608 for further detail.

The active vitamin D compound or the mimic thereof is preferably administered at a dose of about 0.5 μg to about 300 μg, more preferably from about 15 μg to about 200 μg. In a specific embodiment, an effective amount of an active vitamin D compound or a mimic thereof is 3, 4, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 μg or more. In certain embodiments, an effective dose of an active vitamin D compound, or a mimic thereof, is between about 3 μg to about 300 μg, more preferably between about 15 μg to about 260 μg, more preferably between about 30 μg to about 240 μg, more preferably between about 50 μg to about 220 μg, more preferably between about 75 μg to about 200 μg. In another embodiment, an effective amount of an active vitamin D compound, or a mimic thereof, is about 300, 400, 500, 600, 700, 800, 900 μg, 1, 2, 3, 4 or 5 mg. In certain embodiments, an effective dose of an active vitamin D compound, or a mimic thereof, is between about 300 μg to about 5 mg, more preferably between about 500 μg and about 4 mg, more preferably between about 800 μg and about 3 mg, more preferably between about 1 and about 3 mg. In certain embodiments, the methods of the invention comprise administering an active vitamin D compound or a mimic thereof in a dose of about 0.12 μg/kg bodyweight to about 3 μg/kg bodyweight. The compound may be administered by any route, including oral, intramuscular, intravenous, parenteral, rectal, nasal, topical, or transdermal.

If the active vitamin D compound or the mimic thereof is to be administered daily, the dose may be kept low, for example about 0.5 μg to about 5 μg, in order to avoid or diminish the induction of hypercalcemia. If the active vitamin D compound or the mimic thereof has a reduced hypercalcemic effect a higher daily dose may be administered without resulting in hypercalcemia, for example about 10 μg to about 20 μg or higher (up to about 50 μg to about 100 μg).

In a preferred embodiment of the invention, the active vitamin D compound or the mimic thereof is administered by HDPA so that high doses of the active vitamin D compound or the mimic thereof can be administered without inducing hypercalcemia. HDPA refers to intermittently administering an active vitamin D compound or a mimic thereof on either a continuous intermittent dosing schedule or a non-continuous intermittent dosing schedule. High doses of active vitamin D compounds, or a mimic thereof, include doses greater than about 3 μg as discussed in the sections above. Therefore, in certain embodiments of the invention, the methods for the prevention, treatment, or amelioration of thrombosis encompass intermittently administering high doses of active vitamin D compounds or mimics thereof. The frequency of the HDPA can be limited by a number of factors including, but not limited to, the pharmacokinetic parameters of the compound or formulation and the pharmacodynamic effects of the active vitamin D compound or the mimic thereof on the animal. For example, animals having impaired renal function may require less frequent administration of the active vitamin D compound, or the mimic thereof, because of the decreased ability of those animals to excrete calcium.

The following is exemplary only and merely serves to illustrate that the term HDPA can encompass any discontinuous administration regimen designed by a person of skill in the art.

In one example, the active vitamin D compound or the mimic thereof can be administered not more than once every three days, every four days, every five days, every six days, every seven days, every eight days, every nine days, or every ten days. The administration can continue for one, two, three, or four weeks or one, two, or three months, or longer. Optionally, after a period of rest, the active vitamin D compound or the mimic thereof can be administered under the same or a different schedule. The period of rest can be one, two, three, or four weeks, or longer, according to the pharmacodynamic effects of the active vitamin D compound or the mimic thereof on the animal.

In another example, the active vitamin D compound or the mimic thereof can be administered once per week for three months.

In a preferred embodiment, the vitamin D compound or the mimic thereof can be administered once per week for three weeks of a four week cycle. After a one week period of rest, the active vitamin D compound or the mimic thereof can be administered under the same or different schedule.

Further examples of dosing schedules that can be used in the methods of the present invention are provided in U.S. Pat. No. 6,521,608, which is incorporated by reference in its entirety.

The above-described administration schedules are provided for illustrative purposes only and should not be considered limiting. A person of skill in the art will readily understand that all active vitamin D compounds or mimics thereof are within the scope of the invention and that the exact dosing and schedule of administration of the active vitamin D compounds or mimics thereof can vary due to many factors.

The amount of a therapeutically effective dose of a pharmaceutical agent in the acute or chronic management of a disease or disorder may differ depending on factors including, but not limited to, the disease or disorder treated, the specific pharmaceutical agents and the route of administration. According to the methods of the invention, an effective dose of an active vitamin D compound, or a mimic thereof, is any dose of the compound effective to prevent, treat, or ameliorate thrombosis. A high dose of an active vitamin D compound or a mimic thereof can be a dose from about 3 μg to about 300 μg or any dose within this range as discussed above. The dose, dose frequency, duration, or any combination thereof, may also vary according to age, body weight, response, and the past medical history of the animal as well as the route of administration, pharmacokinetics, and pharmacodynamic effects of the pharmaceutical agents. These factors are routinely considered by one of skill in the art.

The rate of absorption and clearance of vitamin D compounds or mimics thereof are affected by a variety of factors that are well known to persons of skill in the art. As discussed above, the pharmacokinetic properties of active vitamin D compounds limit the peak concentration of vitamin D compounds that can be obtained in the blood without inducing the onset of hypercalcemia. The rate and extent of absorption, distribution, binding or localization in tissues, biotransformation, and excretion of the active vitamin D compound can all affect the frequency at which the pharmaceutical agents can be administered.

In one embodiment of the invention, an active vitamin D compound or a mimic thereof is administered at a dose sufficient to achieve peak plasma concentrations of the active vitamin D compound, or the mimic thereof, of about 0.1 nM to about 25 nM. In certain embodiments, the methods of the invention comprise administering the active vitamin D compound or the mimic thereof in a dose that achieves peak plasma concentrations of 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 12.5 nM, 15 nM, 17.5 nM, 20 nM, 22.5 nM, or 25 nM or any range of concentrations therein. In other embodiments, the active vitamin D compound or the mimic thereof is administered in a dose that achieves peak plasma concentrations of the active vitamin D compound or the mimic thereof exceeding about 0.5 nM, preferably about 0.5 nM to about 25 nM, more preferably about 5 nM to about 20 nM, and even more preferably about 10 nM to about 15 nM.

In another preferred embodiment, the active vitamin D compound or the mimic thereof is administered at a dose of at least about 0.12 μg/kg bodyweight, more preferably at a dose of at least about 0.5 μg/kg bodyweight.

One of skill in the art will recognize that these standard doses are for an average sized adult of approximately 70 kg and can be adjusted for the factors routinely considered as stated above.

In certain embodiments, the methods of the invention further comprise administering a dose of an active vitamin D compound, or a mimic thereof, that achieves peak plasma concentrations rapidly, e.g., within four hours. In further embodiments, the methods of the invention comprise administering a dose of an active vitamin D compound, or a mimic thereof, that is eliminated quickly, e.g., with an elimination half-life of less than 12 hours.

While obtaining high concentrations of the active vitamin D compound or the mimic thereof is beneficial, it must be balanced with clinical safety, e.g., hypercalcemia. Thus, in one aspect of the invention, the methods of the invention encompass HDPA of active vitamin D compounds or mimics thereof to an animal before, during, or after angioplasty or bypass surgery and monitoring the animal for symptoms associated with hypercalcemia. Such symptoms include calcification of soft tissues (e.g., cardiac tissue), increased bone density, and hypercalcemic nephropathy. In still another embodiment, the methods of the invention encompass HDPA of an active vitamin D compound, or the mimic thereof, to an animal before, during, or after angioplasty or bypass surgery and monitoring the calcium plasma concentration of the animal to ensure that the calcium plasma concentration is less than about 10.2 mg/dL.

In certain embodiments, high blood levels of vitamin D compounds or mimics thereof can be safely obtained in conjunction with reducing the transport of calcium into the blood. In one embodiment, higher concentrations of active vitamin D compound or mimic thereof are safely obtainable without the onset of hypercalcemia when administered in conjunction with a reduced calcium diet. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., zoledronate, pamidronate, or alendronate, or a corticosteroid such as, e.g., dexamethasone or prednisone, in conjunction with the active vitamin D compound or the mimic thereof.

In certain embodiments, high blood levels of active vitamin D compounds or mimics thereof are safely obtained in conjunction with maximizing the rate of clearance of calcium. In one example, calcium excretion can be increased by ensuring adequate hydration and salt intake. In another example, diuretic therapy can be used to increase calcium excretion.

When the active vitamin D compound or the mimic thereof is delivered locally, e.g., as a coating on a stent, blood levels of active vitamin D compound or calcium do not need to be monitored as the localized delivery is unlikely to result in systemically detectable levels of the active vitamin D compound or to affect systemic calcium levels.

The active vitamin D compound or the mimic thereof may be administered as part of a pharmaceutical composition comprising a pharmaceutically acceptable carrier, wherein the active vitamin D compound or the mimic thereof is present in an amount which is effective to achieve its intended purpose, i.e., to have an anti-thrombotic effect. The pharmaceutical composition may further comprise one or more excipients, diluents or any other components known to persons of skill in the art and germane to the methods of formulation of the present invention. The pharmaceutical composition may additionally comprise other compounds typically used as adjuncts during prevention, treatment, or amelioration of thrombosis.

The term “pharmaceutical composition” as used herein is to be understood as defining compositions of which the individual components or ingredients are themselves pharmaceutically acceptable, e.g., where oral administration is foreseen, acceptable for oral use and, where topical administration is foreseen, topically acceptable.

The pharmaceutical composition can be prepared in single unit dosage forms. The dosage forms are suitable for oral, mucosal (nasal, sublingual, vaginal, buccal, rectal), parenteral (intravenous, intramuscular, intraarterial), or topical administration. Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

In a preferred embodiment of the invention, the pharmaceutical compositions comprising active vitamin D compounds, or mimics thereof, are emulsion pre-concentrate formulations. The compositions of the invention meet or substantially reduce the difficulties associated with active vitamin D compound therapy hitherto encountered in the art including, in particular, undesirable pharmacokinetic parameters of the compound upon administration to a patient.

According to one aspect of the present invention, a pharmaceutical composition is provided comprising (a) a lipophilic phase component, (b) one or more surfactants, (c) an active vitamin D compound or a mimic thereof; wherein said composition is an emulsion pre-concentrate, which upon dilution with water, in a water to composition ratio of about 1:1 or more of said water, forms an emulsion having an absorbance of greater than 0.3 at 400 nm. The pharmaceutical composition of the invention may further comprise a hydrophilic phase component.

In another aspect of the invention, a pharmaceutical emulsion composition is provided comprising water (or other aqueous solution) and an emulsion pre-concentrate.

The term “emulsion pre-concentrate,” as used herein, is intended to mean a system capable of providing an emulsion upon contacting with, e.g., water. The term “emulsion,” as used herein, is intended to mean a colloidal dispersion comprising water and organic components including hydrophobic (lipophilic) organic components. The term “emulsion” is intended to encompass both conventional emulsions, as understood by those skilled in the art, as well as “sub-micron droplet emulsions,” as defined immediately below.

The term “sub-micron droplet emulsion,” as used herein is intended to mean a dispersion comprising water and organic components including hydrophobic (lipophilic) organic components, wherein the droplets or particles formed from the organic components have an average maximum dimension of less than about 1000 nm.

Sub-micron droplet emulsions are identifiable as possessing one or more of the following characteristics. They are formed spontaneously or substantially spontaneously when their components are brought into contact, that is without substantial energy supply, e.g., in the absence of heating or the use of high shear equipment or other substantial agitation. They exhibit thermodynamic stability and they are monophasic.

The particles of a sub-micron droplet emulsion may be spherical, though other structures are feasible, e.g. liquid crystals with lamellar, hexagonal or isotropic symmetries. Generally, sub-micron droplet emulsions comprise droplets or particles having a maximum dimension (e.g., average diameter) of between about 50 nm to about 1000 nm, and preferably between about 200 nm to about 300 nm.

The pharmaceutical compositions of the present invention will generally form an emulsion upon dilution with water. The emulsion will form according to the present invention upon the dilution of an emulsion pre-concentrate with water in a water to composition ratio of about 1:1 or more of said water. According to the present invention, the ratio of water to composition can be, e.g., between 1:1 and 5000:1. For example, the ratio of water to composition can be about 1:1, 2:1, 3:1, 4:1, 5:1, 10:1, 200:1, 300:1, 500:1, 1000:1, or 5000:1. The skilled artisan will be able to readily ascertain the particular ratio of water to composition that is appropriate for any given situation or circumstance.

According to the present invention, upon dilution of said emulsion pre-concentrate with water, an emulsion will form having an absorbance of greater than 0.3 at 400 nm. The absorbance at 400 nm of the emulsions formed upon 1:100 dilution of the emulsion pre-concentrates of the present invention can be, e.g., between 0.3 and 4.0. For example, the absorbance at 400 nm can be about 0.4, 0.5, 0.6, 1.0, 1.2, 1.6, 2.0, 2.2, 2.4, 2.5, 3.0, or 4.0. Methods for determining the absorbance of a liquid solution are well known by those in the art. The skilled artisan will be able to ascertain and adjust the relative proportions of the ingredients of the emulsion pre-concentrates of the invention in order to obtain, upon dilution with water, an emulsion having any particular absorbance encompassed within the scope of the invention.

The pharmaceutical compositions of the present invention can be, e.g., in a solid, semi-solid, or liquid formulation. Semi-solid formulations of the present invention can be any semi-solid formulation known by those of ordinary skill in the art, including, e.g., gels, pastes, creams and ointments.

The pharmaceutical compositions of the present invention comprise a lipophilic phase component. Suitable components for use as lipophilic phase components include any pharmaceutically acceptable solvent which is non-miscible with water. Such solvents will appropriately be devoid or substantially devoid of surfactant function.

The lipophilic phase component may comprise mono-, di- or triglycerides. Mono-, di- and triglycerides that may be used within the scope of the invention include those that are derived from C₆, C₈, C₁₀, C₁₂, C₁₄, C₁₆, C₁₈, C₂₀ and C₂₂ fatty acids. Exemplary diglycerides include, in particular, diolein, dipalmitolein, and mixed caprylin-caprin diglycerides. Preferred triglycerides include vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

Among the above-listed triglycerides, preferred triglycerides include: almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

A preferred triglyceride is the medium chain triglyceride available under the trade name LABRAFAC CC. Other preferred triglycerides include neutral oils, e.g., neutral plant oils, in particular fractionated coconut oils such as known and commercially available under the trade name MIGLYOL, including the products: MIGLYOL 810; MIGLYOL 812; MIGLYOL 818; and CAPTEX 355.

Also suitable are caprylic-capric acid triglycerides such as known and commercially available under the trade name MYRITOL, including the product MYRITOL 813. Further suitable products of this class are CAPMUL MCT, CAPTEX 200, CAPTEX 300, CAPTEX 800, NEOBEE M5 and MAZOL 1400.

Especially preferred as lipophilic phase component is the product MIGLYOL 812. (See U.S. Pat. No. 5,342,625).

Pharmaceutical compositions of the present invention may further comprise a hydrophilic phase component. The hydrophilic phase component may comprise, e.g., a pharmaceutically acceptable C₁₋₅ alkyl or tetrahydrofurfuryl di- or partial-ether of a low molecular weight mono- or poly-oxy-alkanediol. Suitable hydrophilic phase components include, e.g., di- or partial-, especially partial-, -ethers of mono- or poly-, especially mono- or di-, -oxy-alkanediols comprising from 2 to 12, especially 4 carbon atoms. Preferably the mono- or poly-oxy-alkanediol moiety is straight-chained. Exemplary hydrophilic phase components for use in relation to the present invention are those known and commercially available under the trade names TRANSCUTOL and COLYCOFUROL. (See U.S. Pat. No. 5,342,625).

In an especially preferred embodiment, the hydrophilic phase component comprises 1,2-propyleneglycol.

The hydrophilic phase component of the present invention may of course additionally include one or more additional ingredients. Preferably, however, any additional ingredients will comprise materials in which the active vitamin D compound or the mimic thereof is sufficiently soluble, such that the efficacy of the hydrophilic phase as a carrier medium for an active vitamin D compound or a mimic thereof is not materially impaired. Examples of possible additional hydrophilic phase components include lower (e.g., C₁₋₅) alkanols, in particular ethanol.

Pharmaceutical compositions of the present invention also comprise one or more surfactants. Surfactants that can be used in conjunction with the present invention include hydrophilic or lipophilic surfactants, or mixtures thereof. Especially preferred are non-ionic hydrophilic and non-ionic lipophilic surfactants.

Suitable hydrophilic surfactants include reaction products of natural or hydrogenated vegetable oils and ethylene glycol, i.e. polyoxyethylene glycolated natural or hydrogenated vegetable oils, for example polyoxyethylene glycolated natural or hydrogenated castor oils. Such products may be obtained in known manner, e.g., by reaction of a natural or hydrogenated castor oil or fractions thereof with ethylene oxide, e.g., in a molar ratio of from about 1:35 to about 1:60, with optional removal of free polyethyleneglycol components from the product, e.g., in accordance with the methods disclosed in German Auslegeschriften 1,182,388 and 1,518,819.

Suitable hydrophilic surfactants for use in the present pharmaceutical compounds also include polyoxyethylene-sorbitan-fatty acid esters, e.g., mono- and trilauryl, palmityl, stearyl and oleyl esters, e.g., of the type known and commercially available under the trade name TWEEN; including the products:

TWEEN 20 (polyoxyethylene(20)sorbitanmonolaurate),

TWEEN 40 (polyoxyethylene(20)sorbitanmonopalmitate),

TWEEN 60 (polyoxyethylene(20)sorbitanmonostearate),

TWEEN 80 (polyoxyethylene(20)sorbitanmonooleate),

TWEEN 65 (polyoxyethylene(20)sorbitantristearate),

TWEEN 85 (polyoxyethylene(20)sorbitantrioleate),

TWEEN 21 (polyoxyethylene(4)sorbitanmonolaurate),

TWEEN 61 (polyoxyethylene(4)sorbitanmonostearate), and

TWEEN 81 (polyoxyethylene(5)sorbitanmonooleate).

Especially preferred products of this class for use in the compositions of the invention are the above products TWEEN 40 and TWEEN 80. (See Hauer, et al., U.S. Pat. No. 5,342,625).

Also suitable as hydrophilic surfactants for use in the present pharmaceutical compounds are polyoxyethylene alkylethers; polyoxyethylene glycol fatty acid esters, for example polyoxyethylene stearic acid esters; polyglycerol fatty acid esters; polyoxyethylene glycerides; polyoxyethylene vegetable oils; polyoxyethylene hydrogenated vegetable oils; reaction mixtures of polyols and, e.g., fatty acids, glycerides, vegetable oils, hydrogenated vegetable oils, and sterols; polyoxyethylene-polyoxypropylene co-polymers; polyoxyethylene-polyoxypropylene block co-polymers; dioctylsuccinate, dioctylsodiumsulfosuccinate, di-[2-ethylhexyl]-succinate or sodium lauryl sulfate; phospholipids, in particular lecithins such as, e.g., soya bean lecithins; propylene glycol mono- and di-fatty acid esters such as, e.g., propylene glycol dicaprylate, propylene glycol dilaurate, propylene glycol hydroxystearate, propylene glycol isostearate, propylene glycol laurate, propylene glycol ricinoleate, propylene glycol stearate, and, especially preferred, propylene glycol caprylic-capric acid diester; and bile salts, e.g., alkali metal salts, for example sodium taurocholate.

Suitable lipophilic surfactants include alcohols; polyoxyethylene alkylethers; fatty acids; bile acids; glycerol fatty acid esters; acetylated glycerol fatty acid esters; lower alcohol fatty acids esters; polyethylene glycol fatty acids esters; polyethylene glycol glycerol fatty acid esters; polypropylene glycol fatty acid esters; polyoxyethylene glycerides; lactic acid esters of mono/diglycerides; propylene glycol diglycerides; sorbitan fatty acid esters; polyoxyethylene sorbitan fatty acid esters; polyoxyethylene-polyoxypropylene block copolymers; trans-esterified vegetable oils; sterols; sugar esters; sugar ethers; sucroglycerides; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; and mixtures thereof.

Suitable lipophilic surfactants for use in the present pharmaceutical compounds also include trans-esterification products of natural vegetable oil triglycerides and polyalkylene polyols. Such trans-esterification products are known in the art and may be obtained e.g., in accordance with the general procedures described in U.S. Pat. No. 3,288,824. They include trans-esterification products of various natural (e.g., non-hydrogenated) vegetable oils for example, maize oil, kernel oil, almond oil, ground nut oil, olive oil and palm oil and mixtures thereof with polyethylene glycols, in particular polyethylene glycols having an average molecular weight of from 200 to 800. Preferred are products obtained by trans-esterification of 2 molar parts of a natural vegetable oil triglyceride with one molar part of polyethylene glycol (e.g., having an average molecular weight of from 200 to 800). Various forms of trans-esterification products of the defined class are known and commercially available under the trade name LABRAFIL.

Additional lipophilic surfactants that are suitable for use with the present pharmaceutical compositions include oil-soluble vitamin derivatives, e.g., tocopherol PEG-1000 succinate (“vitamin E TPGS”).

Also suitable as lipophilic surfactants for use in the present pharmaceutical compounds are mono-, di- and mono/di-glycerides, especially esterification products of caprylic or capric acid with glycerol; sorbitan fatty acid esters; pentaerythritol fatty acid esters and polyalkylene glycol ethers, for example pentaerythrite-dioleate, -distearate, -monolaurate, -polyglycol ether and -monostearate as well as pentaerythrite-fatty acid esters; monoglycerides, e.g., glycerol monooleate, glycerol monopalmitate and glycerol monostearate; glycerol triacetate or (1,2,3)-triacetin; and sterols and derivatives thereof, for example cholesterols and derivatives thereof, in particular phytosterols, e.g., products comprising sitosterol, campesterol or stigmasterol, and ethylene oxide adducts thereof, for example soya sterols and derivatives thereof.

It is understood by those of ordinary skill in the art that several commercial surfactant compositions contain small to moderate amounts of triglycerides, typically as a result of incomplete reaction of a triglyceride starting material in, for example, a trans-esterification reaction. Thus, the surfactants that are suitable for use in the present pharmaceutical compositions include those surfactants that contain a triglyceride. Examples of commercial surfactant compositions containing triglycerides include some members of the surfactant families GELUCIRES, MAISINES, and IMWITORS. Specific examples of these compounds are GELUCIRE 44/14 (saturated polyglycolized glycerides); GELUCIRE 50/13 (saturated polyglycolized glycerides); GELUCIRE 53/10 (saturated polyglycolized glycerides); GELUCIRE 33/01 (semi-synthetic triglycerides of C₈-C₁₈ saturated fatty acids); GELUCIRE 39/01 (semi-synthetic glycerides); other GELUCIRES, such as 37/06, 43/01, 35/10, 37/02, 46/07, 48/09, 50/02, 62/05, etc.; MAISINE 35-I (linoleic glycerides); and IMWITOR 742 (caprylic/capric glycerides). (See U.S. Pat. No. 6,267,985).

Still other commercial surfactant compositions having significant triglyceride content are known to those skilled in the art. It should be appreciated that such compositions, which contain triglycerides as well as surfactants, may be suitable to provide all or part of the lipophilic phase component of the of the present invention, as well as all or part of the surfactants.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative of preferred or individually inventive teachings only and not as limiting the invention in its broadest aspect.

The lipophilic phase component of the invention will suitably be present in an amount of from about 30% to about 90% by weight based upon the total weight of the composition. Preferably, the lipophilic phase component is present in an amount of from about 50% to about 85% by weight based upon the total weight of the composition.

The surfactant or surfactants of the invention will suitably be present in an amount of from about 1% to 50% by weight based upon the total weight of the composition. Preferably, the surfactant(s) is present in an amount of from about 5% to about 40% by weight based upon the total weight of the composition.

The amount of active vitamin D compound or mimic thereof in compositions of the invention will of course vary, e.g., depending on the intended route of administration and to what extent other components are present. In general, however, the active vitamin D compound, or the mimic thereof, of the invention will suitably be present in an amount of from about 0.005% to 20% by weight based upon the total weight of the composition. Preferably, the active vitamin D compound or the mimic thereof is present in an amount of from about 0.01% to 15% by weight based upon the total weight of the composition.

The hydrophilic phase component of the invention will suitably be present in an amount of from about 2% to about 20% by weight based upon the total weight of the composition. Preferably, the hydrophilic phase component is present in an amount of from about 5% to 15% by weight based upon the total weight of the composition.

The pharmaceutical composition of the invention may be in a semisolid formulation. Semisolid formulations within the scope of the invention may comprise, e.g., a lipophilic phase component present in an amount of from about 60% to about 80% by weight based upon the total weight of the composition, a surfactant present in an amount of from about 5% to about 35% by weight based upon the total weight of the composition, and an active vitamin D compound or a mimic thereof present in an amount of from about 0.01% to about 15% by weight based upon the total weight of the composition.

The pharmaceutical compositions of the invention may be in a liquid formulation. Liquid formulations within the scope of the invention may comprise, e.g., a lipophilic phase component present in an amount of from about 50% to about 60% by weight based upon the total weight of the composition, a surfactant present in an amount of from about 4% to about 25% by weight based upon the total weight of the composition, an active vitamin D compound, or a mimic thereof, present in an amount of from about 0.01% to about 15% by weight based upon the total weight of the composition, and a hydrophilic phase component present in an amount of from about 5% to about 10% by weight based upon the total weight of the composition.

Additional compositions that may be used include the following, wherein the percentage of each component is by weight based upon the total weight of the composition excluding the active vitamin D compound or the mimic thereof:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 50%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound or a mimic thereof, a lipophilic component, and a surfactant. The lipophilic component may be present in any percentage from about 1% to about 100%. The lipophilic component may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise 50% MIGLYOL 812 and 50% vitamin E TPGS, 90% MIGLYOL 812 and 10% vitamin E TPGS, or 95% MIGLYOL 812 and 5% vitamin E TPGS.

In another embodiment of the invention, the pharmaceutical compositions comprise an active vitamin D compound, or a mimic thereof, and a lipophilic component, e.g., around 100% MIGLYOL 812.

In a preferred embodiment, the pharmaceutical compositions comprise 50% MIGLYOL 812, 50% vitamin E TPGS, and small amounts of BHA and BHT. This formulation has been shown to be unexpectedly stable, both chemically and physically (see Example 3). The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound or a mimic thereof, thereby enabling high dose pulse administration of active vitamin D compounds, or mimics thereof, for the treatment of hyperproliferative diseases and other disorders.

The pharmaceutical compositions comprising the active vitamin D compound, or the mimic thereof, of the present invention may further comprise one or more additives. Additives that are well known in the art include, e.g., detackifiers, anti-foaming agents, buffering agents, antioxidants (e.g., ascorbyl palmitate, butyl hydroxy anisole (BHA), butyl hydroxy toluene (BHT) and tocopherols, e.g., α-tocopherol (vitamin E)), preservatives, chelating agents, viscomodulators, tonicifiers, flavorants, colorants odorants, opacifiers, suspending agents, binders, fillers, plasticizers, lubricants, and mixtures thereof. The amounts of such additives can be readily determined by one skilled in the art, according to the particular properties desired. For example, antioxidants may be present in an amount of from about 0.05% to about 0.35% by weight based upon the total weight of the composition.

The additive may also comprise a thickening agent. Suitable thickening agents may be those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents as aforesaid will generally not be required and is generally less preferred. Use of thickening agents is, on the other hand, indicated, e.g., where topical application is foreseen.

Compositions in accordance with the present invention may be employed for administration in any appropriate manner, e.g., orally, e.g., in unit dosage form, for example in a solution, in hard or soft encapsulated form including gelatin encapsulated form, parenterally or topically, e.g., for application to the skin, for example in the form of a cream, paste, lotion, gel, ointment, poultice, cataplasm, plaster, dermal patch or the like, as a coating for a medical device, e.g., a stent, or for ophthalmic application, for example in the form of an eye-drop, -lotion or -gel formulation. Readily flowable forms, for example solutions and emulsions, may also be employed e.g., for intralesional injection, or may be administered rectally, e.g., as an enema.

When the composition of the present invention is formulated in unit dosage form, the active vitamin D compound or the mimic thereof will preferably be present in an amount of between 1 and 200 μg per unit dose. More preferably, the amount of active vitamin D compound or the mimic thereof per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μg or any amount therein. In a preferred embodiment, the amount of active vitamin D compound or the mimic thereof per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

Animals which may be treated according to the present invention include all animals which may benefit from administration of the compounds of the present invention. Such animals include humans, pets such as dogs and cats, and veterinary animals such as cows, pigs, sheep, goats and the like.

The following examples are illustrative, but not limiting, of the methods of the present invention. Other suitable modifications and adaptations of the variety of conditions and parameters normally encountered in medical treatment and pharmaceutical science and which are obvious to those skilled in the art are within the spirit and scope of the invention.

### EXAMPLE 1

### Preparation of Semi-solid Calcitriol Formulations

Five semi-solid calcitriol formulations (SS1—SS5) were prepared containing the ingredients listed in Table 1. The final formulation contains 0.208 mg calcitriol per gram of semi-solid formulation.

TABLE 1
Composition of Semi-Solid Calcitriol Formulation

| Ingredients | SS1 | SS2 | SS3 | SS4 | SS5 |
|---|---|---|---|---|---|
| Calcitriol | 0.0208 | 0.0208 | 0.0208 | 0.0208 | 0.0208 |
| Miglyol 812 | 80.0 | 0 | 65.0 | 0 | 79.0 |
| Captex 200 | 0 | 82.0 | 0 | 60.0 | 0 |
| Labrafac CC | 0 | 0 | 0 | 0 | 12.0 |
| Vitamin-E TPGS | 20.0 | 18.0 | 5.0 | 5.0 | 9.0 |
| Labrifil M | 0 | 0 | 0 | 0 | 0 |
| Gelucire 44/14 | 0 | 0 | 30.0 | 35.0 | 0 |
| BHT | 0.05 | 0.05 | 0.05 | 0.05 | 0.05 |
| BHA | 0.05 | 0.05 | 0.05 | 0.05 | 0.05 |

Amounts shown are in grams.

### 1. Preparation of Vehicles

One hundred gram quantities of the five semi-solid calcitriol formulations (SS1—SS5) listed in Table 1 were prepared as follows.

The listed ingredients, except for calcitriol, were combined in a suitable glass container and mixed until homogenous. Vitamin E TPGS and GELUCIRE 44/14 were heated and homogenized at 60° C. prior to weighing and adding into the formulation.

### 2. Preparation of Active Formulations

The semi-solid vehicles were heated and homogenized at <60° C. Under subdued light, 12±1 mg of calcitriol was weighed out into separate glass bottles with screw caps, one bottle for each formulation. (Calcitriol is light sensitive; subdued light/red light should be used when working with calcitriol/calcitriol formulations.) The exact weight was recorded to 0.1 mg. The caps were then placed on the bottles as soon as the calcitriol had been placed into the bottles. Next, the amount of each vehicle required to bring the concentration to 0.208 mg/g was calculated using the following formula:
C_{w}/0.208=required weight of vehicle
Where C_{w}=weight of calcitriol, in mg, and
0.208=final concentration of calcitriol (mg/g).

Finally, the appropriate amount of each vehicle was added to the respective bottle containing the calcitriol. The formulations were heated (<60° C.) while being mixed to dissolve the calcitriol.

### EXAMPLE 2

### Preparation of Additional Formulations

Following the method of Example 1, twelve different formulations for calcitriol were prepared containing the ingredients listed in Table 2.

TABLE 2
Composition Formulations

| IngredIents | 1 | 2 | 3 | 4 | 5 | 6 | 7 | 8 | 9 | 10 | 11 | 12 |
|---|---|---|---|---|---|---|---|---|---|---|---|---|
| Miglyol | 95 | 65 | 90 | 85 | 80 | 95 | 65 | 90 | 85 | 80 | 50 | 0 |
| 812N | | | | | | | | | | | | |
| Vitamin | 5 | 5 | 10 | 5 | 10 | 5 | 5 | 10 | 5 | 10 | 50 | 50 |
| E TPGS | | | | | | | | | | | | |
| PEG | 0 | 30 | 0 | 10 | 10 | 0 | 30 | 0 | 10 | 10 | 0 | 50 |
| 4000 | | | | | | | | | | | | |
| BHA | 0.05 | 0.05 | 0.05 | 0.05 | 0.05 | 0.35 | 0.35 | 0.35 | 0.35 | 0.35 | 0.35 | 0.35 |
| BHT | 0.05 | 0.05 | 0.05 | 0.05 | 0.05 | 0.35 | 0.35 | 0.35 | 0.35 | 0.35 | 0.35 | 0.35 |

Amounts shown are percentages.

### EXAMPLE 3

### Stable Unit Dose Formulations

Formulations of calcitriol were prepared to yield the compositions in Table 3. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 3
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 4, all three formulations produced an opalescent solution upon mixing with water. Formulation 4 appeared to form a stable suspension with no observable change in absorbance at 400 nm after 10 min.

TABLE 4
Absorption of formulations suspended in water

Absorbance at 400 nm

| Formulation # | Initial | 10 min |
|---|---|---|
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 5. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 6).

The chemical stability results indicated that decreasing the MIGLYOL 812 content with a concomitant increase in Vitamin E TPGS content provided enhanced recovery of intact calcitriol as noted in Table 5. Formulation 4 (50:50 MIGLYOL 812/Vitamin E TPGS) was the most chemically stable formulation with only minor decreases in recovery of intact calcitriol after 3 months at 25° C./60% RH, enabling room temperature storage.

TABLE 5
Chemical stability of calcitriol formulation in hard gelatin
capsules (225 mg total mass filled per capsule, 45 μg calcitriol)

| Storage | Time | Assay^{a} (%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 6
Physical Stability of Calcitriol Formulation in Hard Gelatin
Capsules (225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The physical stability of the formulations was assessed by the dissolution behavior of the capsules after storage at each stability condition. As with the chemical stability, decreasing the MIGLYOL 812 content and increasing the Vitamin E TPGS content improved the dissolution properties of the formulation (Table 6). Formulation 4 (50:50 MIGLYOL 812/Vitamin E TPGS) had the best dissolution properties with suitable stability for room temperature storage.

### EXAMPLE 4

### Phase II Clinical Trial

Two hundred fifty patients with prostate cancer were enrolled at 58 centers in the United States and Canada. All patients in the study received chemotherapy treatment with Taxotere®, a drug in the taxoid class of chemotherapeutic agents. Taxotere® is approved for use in prostate cancer and some other types of cancer. Oral dexamethasone is also given along with the Taxotere® to minimize certain side effects (allergic reactions and fluid retention) associated with Taxotere®.

In addition to Taxotere® and dexamethasone, half of the patients were randomly treated with calcitriol and the other half received a placebo. Calcitriol was administered as three capsules of 15 μg each once a week on the day prior to chemotherapy. Previous studies in more than 90 cancer patients suggest that weekly dosing allows patients to receive high doses of calcitriol while minimizing the side effect of high blood calcium (hypercalcemia). The same Taxotere® doses of 75 mg/m² body surface area were administered to the patients receiving Taxotere® alone or Taxotere® in combination with calcitriol.

Patients receiving Taxotere® and calcitriol by HDPA experienced fewer cardiovascular events compared to patients treated with Taxotere® without calcitriol. These cardiovascular events include cerebrovascular events or stroke where two of 125 patients treated with Taxotere® alone had a stroke while none of 125 patients treated with Taxotere® and calcitriol by HDPA suffered a stroke. Moreover, six of 125 patients who did not receive calcitriol developed deep vein thrombosis or thrombophlebits while two of the 125 patients treated with calcitriol by HDPA developed the condition. In addition, two of 125 patients treated with Taxotere® alone developed myocardial infractions or myocardial ischemia while none of the 125 patients treated with Taxotere® and calcitriol by HDPA did.

### EXAMPLE 5

### Calcitriol in Combination AVASTIN® and One or More Chemotherapeutic Agents

The active vitamin D compound or the mimic thereof will be tested in combination with AVASTIN® as a first-line treatment of metastatic carcinoma of the colon or rectum. Patients will be randomized to bolus-IFL (iritotecan 125 mg/m² IV, 5-fluoruuracil 500 mg/m² IV and leucovorin 20 mg/m² IV given once weekly on day 2 for 4 weeks every 6 weeks) plus placebo (Arm 1), bolus-IFL plus AVASTIN® (5 mg/kg every two weeks on day 2) (Arm 2), 5-FU/LV (5-fluoruuracil 500 mg/m² IV and leucovorin 20 mg/m² IV given once weekly on day 2 for 4 weeks every 6 weeks) plus AVASTIN® (5 mg/kg every two weeks on day 2) (Arm 3), bolus-IFL plus AVASTIN® (5 mg/kg every two weeks on day 2) plus calcitriol (45 μg once weekly on day 1) (Arm 4), and 5-FU/LV plus AVASTIN® (5 mg/kg every two weeks on day 2) plus calcitriol (45 μg once weekly on day 1) (Arm 5). To optimize the efficacy of calcitriol to treat, prevent, or ameliorate thrombotic disorders associated with the administration of AVASTIN® in combination with the chemotherapeutic agents, calcitriol dosage will be varied from 5 μg to about 180 μg, administered once a week on the day prior to the administration of AVASTIN® and the chemotherapeutic agents.

Cardiovascular events exhibited by patients receiving calcitriol by HDPA will be compared to those events exhibited by patients treated with AVASTIN® in combination with bolus-IFL or 5-FU/LV. Other chemotherapeutic agents will similarly be tested in combination with an active vitamin D compound or a mimic thereof and AVASTIN®.

Those skilled in the art can design additional trial protocols suitable for optimizing the efficacy of calcitriol to treat, prevent, or ameliorate thrombotic disorders associated with the administration of AVASTIN® in combination with one or more chemotherapeutic agents.

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method for preventing, treating or ameliorating a thrombotic disorder comprising administering by high dose pulse administration (HDPA) to an animal in need of such a treatment a pharmaceutical composition comprising an effective amount of active vitamin D compound or a mimic thereof.

2. The method of claim 1, wherein said active vitamin D compound is selected from the group consisting of calcitriol, 1α-calcidol and calcifediol.

3. The method of claim 1, wherein said active vitamin D compound is calcitriol.

4. The method of claim 1, wherein said active vitamin D compound is 1α-calcidol.

5. The method of claim 1, wherein said active vitamin D compound is calcifediol.

6. The method of claim 2, wherein said active vitamin D compound is administered as a unit dosage form comprising about 10 μg to about 75 μg of calcitriol, about 50% MIGLYOL 812 and about 50% tocopherol PEG-1000 succinate (vitamin E TPGS).

7. The method of claim 6, wherein said active vitamin D compound is administered as a unit dosage form comprising about 45 μg of calcitriol, about 50% MIGLYOL 812, about 50% vitamin E TPGS, BHA, and BHT.

8. The method of claim 6, wherein said unit dosage form is a capsule wherein the total volume of ingredients in said capsule is between about 10 μL to about 1000 μL.

9. The method of claim 1, wherein said HDPA is administered no more frequently than once in three days.

10. The method of claim 1, wherein said active vitamin D compound is administered orally, intravenously, parenterally, rectally, sublingually, intramuscularly, topically, nasally or transdermally.

11. The method of claim 1, further comprising administering one or more therapeutic agents.

12. The method of claim 11, wherein said one or more therapeutic agent is a chemotherapeutic agent, an anti-angiogenic factor or a combination thereof.

13. The method of claim 11, wherein said one or more therapeutic agents are a contributing cause to said thrombosis.

14. The method of claim 13, wherein said one or more therapeutic agents are selected from the group consisting of anti-angiogenic factors, vasodilators, immunosuppressants, anti-inflammatories, and collagen synthetase inhibitors, actinomycin D, irinotecan, vincristine, vinblastine, vinorelbine, SN-38, azacitidine, thalidomide, methotrexate, azathioprine, fluorouracil, doxorubicin, mitomycin, nitrates, calcium channel blockers, hirudin, iloprost, sirolimus, everolimus, A24, tranilast, dexamethasone, tacrolimus, halofuginone, propyl hydroxylase, C-proteinase inhibitor, metalloproteinase inhibitor, corticosteroids, non-steroidal anti-inflammatory drugs, 17β-estradiol, angiotensin converting enzyme inhibitors, colchicine, fibroblast growth factor antagonists, histamine antagonists, lovastatin, nitroprusside, phosphodiesterase inhibitors, prostaglandin inhibitors, suramin, serotonin blockers, thioprotease inhibitors, platelet-derived growth factor antagonists, nitric oxide, angiopeptin and antineoplastic agents comprising paclitaxel and docetaxel.

15. The method of claim 12 or 14, wherein said anti-angiogenic factor is selected from the group consisting of bevacizumab, VEGF-TRAP, anti-VEGF-receptor antibodies, angiostatin, endostatin, batimastat, captopril, cartilage derived inhibitor, genistein, interleukin 12, lavendustin, medroxypregesterone acetate, recombinant human platelet factor 4, tecogalan, thrombospondin, TNP-470, VEGF antagonists, anti-VEGF monoclonal antibodies, soluble VEGF-receptor chimaeric proteins, antisense oligonucleotides, antisense oligodexoynucleotides, siRNAs, anti-VEGF aptamers, pigment epithelium derived factor, tyrosine kinase inhibitors, inhibitors of epidermal-derived growth factor, inhibitors of fibroblast-derived growth factor, inhibitors of platelet derived growth factor, matrix metalloprotease inhibitors, integrin blockers, interferon-α, pentosan polysulfate, cyclooxygenase inhibitors, carboxyamidotriazole, combretastatin A-4, squalamine, 6-O-chloroacetyl-carbonyl)-fumagillol, thalidomide, troponin-1, indolinethiones, pyridopyrimidines, quinoazolines, phenyl-pyrrolo-pyrimidines, trastuzumab, calcium influx inhibitors, neomycin, squalamine, marimastat, prinomastat, metastat and cinnoline derivatives.

16. The method of claim 11, wherein said one or more therapeutic agents are anti-thrombotic agents.

17. The method of claim 16, wherein said one or more therapeutic agents are selected from the group consisting of anticoagulants, anti-platelet agents, anti-thrombins, heparin, aspirin, blockers of IIb/IIa receptors hirudin, platelet-derived growth factor antagonists, coumarin, bishydroxycoumarin, warfarin, acid citrate dextrose, lepirudin, ticlopidine, clopidogrel, tirofiban, argatroban, and eptifibatide.

18. The method of claim 11, wherein said one or more therapeutic agents is a taxane.

19. The method of claim 18, wherein said taxane is paclitaxel or docetaxel.

20. The method of claim 1, wherein said thrombotic disorder is selected from the group consisting of venous and arterial thrombosis, congestive heart failure, transient ischemic attacks, stroke, pulmonary embolism, arterial embolism, atherosclerosis, myocardial ischemia, myocardial infarction, cerebral thrombosis and ischemia, atherosclerosis and arteriosclerosis, angina, peripheral vascular disease, preeclampsia, and restenosis following angioplasty, carotid endarterectomy or anastomosis of vascular grafts.
